SEQUENCE LISTING (3 sequences)

SEQ ID NO: 1 - protein, 9 aa, synthetic construct
  VARIANT 2..3: note = Xaa can be any naturally occurring amino acid
  VARIANT 6: note = Xaa can be any naturally occurring amino acid
RXXRRXRRR

SEQ ID NO: 2 - protein, 9 aa, synthetic construct
RKKRRQRRR

SEQ ID NO: 3 - protein, 12 aa, synthetic construct
GRKKRRQRRRPQ

(19) United States
(12) Patent Application Publication
(10) Pub. No.: US 2025/0295795 A1
(43) Pub. Date: Sep. 25, 2025

(54) PEPTIDE

(71) Applicant: CANCER RESEARCH TECHNOLOGY LIMITED, Greater London (GB)

(72) Inventors: Katherine Vallis, Oxford (GB); Ole Tietz, Syndey, New South Wales (AU)

(21) Appl. No.: 18/682,572

(22) PCT Filed: Aug. 11, 2022
(86) PCT No.: PCT/GB2022/052093

§ 371 Date: Feb. 9, 2024

(30) Foreign Application Priority Data
Aug. 13, 2021 (GB) 2111675.1

(51) Int. Cl. A61K 47/64; C07K 7/64

(52) U.S. Cl. CPC A61K 47/645; C07K 7/64

(57) ABSTRACT

A compound comprising formula I; wherein; W is an extension moiety, X is selected from a cargo moiety, or H, Y is a linking moiety, Z is a cell penetrating peptide comprising SEQ ID NO:1 (RXXRRXRRR), wherein n=0 to 10, and wherein Z is joined to Y via one atom of Z, or is joined to Y via at least two atoms of Z such as to form a cyclic moiety with Y. The invention also extends to methods for use of the compound.

### STATEMENT OF PRIORITY

This application is a 35 U.S.C. § 371 national phase application of PCT Application No. PCT/GB2022/052093 filed Aug. 11, 2022, which claims the benefit of and priority to British Application No. 2111675.1 filed Aug. 13, 2021, the entire contents of each of which are incorporated by reference herein.

### STATEMENT REGARDING ELECTRONIC FILING OF A SEQUENCE LISTING

A Sequence Listing in XML format, submitted under 37 C.F.R. § 1.831-1.834, entitled 1553-23_ST26.xml, 4,431 bytes in size, generated on Mar. 23, 2025 and filed electronically, is provided in lieu of a paper copy. This Sequence Listing is hereby incorporated by reference into the specification for its disclosures.

### FIELD OF INVENTION

The present invention relates to trimers of cell penetrating peptides and methods for their use.

### BACKGROUND

Agents that can deliver exogenous cargo into live cells are highly sought after and find wide application in cell biology; furthermore, effective intracellular targeting is crucial in the development of novel diagnostic and therapeutic agents. Intracellular delivery of functional monoclonal antibodies or antibody fragments would allow the utilization of previously undruggable but therapeutically relevant targets, such as the large number of intracellular protein-protein interactions (PPI). Despite advances in the field, intracellular delivery remains a formidable challenge due to the low efficacy or the high toxicity of current vehicles. Polycationic molecules, such as polymers, lipid particles, and cell-penetrating peptides (CPPs), have been studied extensively over the past three decades as a means of transporting pharmacons into cells. The archetypal CPP Tat has been used extensively in the design of intracellularly targeted therapeutics; however, endocytic entrapment is recognized as a significant hindrance to intracellular delivery. Low level leakage of Tat delivered cargos from endosomes is typically too inefficient to lend itself to most intracellular targeting applications.

A number of recent studies have attempted to address this problem (Dougherty, P. G., Sahni, A., Pei, D. Understanding Cell Penetration of Cyclic Peptides. Chem. Rev. 2019). Cyclized Tat and oligoarginine conjugated to GFP (cTat-GFP) were found to access the cytosol and nucleus by direct translocation across the cell membrane, (Nishan, N, et al. Covalent attachment of cyclic TAT peptides to GFP results in protein delivery into live cells with immediate bioavailability. Angew. Chem. Int. Ed. 54, 1950-1953, 2015 and Herce, H. D. et al. Cell-permeable nanobodies for targeted immunolabelling and antigen manipulation in living cells. Nat. Chem. 9, 762-771, 2017), while a CPP specifically designed to be endosomolytic (L17E) was shown to facilitate the escape of cargo from the endosome (Akishiba, M. et al. Cytosolic antibody delivery by lipid-sensitive endosomolytic peptide. Nat. Chem. 9, 751-761, 2017). A significant barrier to utilization of these approaches in translational in vivo applications is the relatively high concentration needed to achieve desirable results in vitro (>20 μM).

### SUMMARY OF INVENTION

The intracellular environment hosts a large number of cancer and other disease relevant human proteins. Targeting these with biomacromolecules would allow therapeutic modulation of hitherto undruggable pathways, such as those mediated by protein-protein interactions (PPI). However, one of the major obstacles in intracellular targeting is the entrapment of biomacromolecules in the endosome.

As such, the present invention relates to an approach for delivering biomacromolecules such as antibodies or antibody fragments into the cytosol and nucleus of cells by using trimeric cell-penetrating peptides (CPP). It has been found that CPP trimers are significantly more potent than CPP monomers and can be tuned to function by direct interaction with the plasma membrane or escape from vesicle like bodies. By using these CPP trimers it is possible to deliver functional biomacromolecules, such as antibodies and Fab fragments, to the cytosol of a cell, whilst maintaining their activity. It has also been shown that the CPP trimers demonstrate this cell delivery activity when the CPPs are in either a linear format or a cyclic format within the trimer.

In a first aspect the invention relates to a compound comprising formula I;

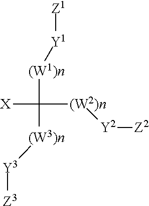

wherein;

W is an extension moiety,

X is selected from a linking moiety, a cargo moiety, a fluorophore, or a H

Y is a linking moiety,

Z is a cell penetrating peptide comprising SEQ ID NO:1 (RXXRRXRRR).

wherein n=0 to 10, and wherein Z is joined to Y via one atom of Z, or is joined to Y via at least two atoms of Z such as to form a cyclic moiety with Y.

In an embodiment the cell penetrating peptide comprises SEQ ID NO:2 (RKKRRQRRR).

Compounds of the invention are trimeric and thus include three cell penetrating peptides.

In an embodiment the cell penetrating peptide is linear. In another embodiment the cell penetrating peptide is cyclic. Examples where the cell penetrating peptide is linear are shown in the molecules of Formula IV and Formula VI. Examples where the cell penetrating peptide is cyclic are shown in Formula V and Formula VII.

In an embodiment the cell penetrating peptide is cyclised via a C terminal glutamic acid and a N-terminal azide-modified lysine or propagyl glycine. In an embodiment the cell penetrating peptide comprises an N-terminus modified with a hexanoyl or an azido-pentanoyl, which is conjugated to the linking moiety. In an embodiment n=0 to 7. In an embodiment n=0 to 5. In an embodiment the extension moiety is selected from (—CH2—), (—CH2—O—CH2—), poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), poly(N-acryloylmorpholine) (PAcM), hyaluronic acid (HA), Heparin, or polysialic acid (PSA). In an embodiment the extension moiety is selected from (—CH2—) or (—CH2—O—CH2—). In an embodiment the extension moiety is selected from (—CH2—) and n=1 to 5. In an embodiment the extension moiety is selected from (—CH2—O—CH2—) and n=1 to 5. In an embodiment the linking moiety comprises a C3-C12 cycloalkyl, C1-C12 heterocycloalkyl, a C3-C12 aryl, a 3-membered to 12-membered heteroaryl. In an embodiment the linking moiety comprises a heterocyclic group, selected from a four-membered ring, a five membered ring, or a six membered ring. In an embodiment the linking moiety comprises 1 to 5 hetero atoms. In an embodiment the cargo moiety is selected from a fluorophore, a small molecule therapeutic, a therapeutic peptide or therapeutic moiety, a protein, an antibody, an antigen-binding fragment, a single chain variable fragment, a single domain antibody, antibody fragment, or an oligonucleotide.

In an embodiment the compound according to the invention is for use as a cell delivery agent.

In an embodiment the compound according to the invention is for use as a co-delivery agent, wherein the co-delivery is of an antibody, or antibody fragment into a cell.

In an embodiment the compound according to the invention is for use as an in vitro diagnostic agent.

In an embodiment the compound according to the invention is for use in therapy. The invention also relates to a method of treating a disease comprising administering a compound described herein to a patient in need thereof. The invention also relates to the use of a compound described herein for the manufacture of a medicament for therapeutic application. In certain embodiments for therapeutic uses, the compound described herein is co-delivered with a therapeutic moiety.

For example, the disease to be treated is selected from a cancer, an inflammatory or an autoimmune disease.

In an embodiment the invention relates to a method of delivering a molecule into a cell comprising contacting a cell with the compound according to the invention.

In an embodiment the invention relates to a method of cyclising a cell penetrating peptide comprising the steps of;
incorporating an azide modified lysine residue or a propagyl glycine to the N-terminus of the Tat peptide,
incorporating a glutamic acid to the C-terminus of the Tat peptide, and performing head-to-tail cyclisation.

### FIGURES

FIG. 1. Synthesis of Trimer Tat constructs, synthesis of tri-Tat A and tri-cTat A (a), tri-Tat B and tri-cTat B (f). In silico generated ball and stick models of linear Tat trimers suggest that they adopt coiled helices which arrange in a more compact conformation in tri-Tat A. Cyclic peptides adopt tighter helices, due to the C-terminus being forced towards the centre of the molecule. In all four trimers, peptides align in an off-parallel fashion, with arginine side chains pointing outwards. (Hydrogens removed for clarity, Carbon—grey, Oxygen—red, Nitrogen—blue, Sulphur—orange; geometry of trimer conjugates optimized using a Dreiding-like forcefield; the peptide backbones highlighted from N-terminus (blue) to C-terminus (red); the central carbon of the tetrakis core highlighted in yellow.) Fluorescence excitation and emission spectra of trimer (black line) compared to AF488 (dotted line); y axis—normalized fluorescence intensity (A.U.); x axis—wavelength (nm).

FIG. 2. Live cell confocal microscopy of linear and cyclic Tat trimers in Hela and CHO cells. (a, f) 1 μM mono-Tat (a) or mono-cTat (f) are not taken up into HeLa or CHO cells. (b, g, h) 1 μM linear tri-Tat A (b) or cyclic tri-cTat A (g) and tri-cTat B (h) are taken up into the cytosol and nucleoli of HeLa and CHO cells. (c) 1 μM linear tri-Tat B is only taken up into endosomes of HeLa and CHO cells. (d, i) Quantification of average fluorescence intensity per cell in cells treated with linear (d) or cyclic (i) constructs. (e, j) Quantification of the percentage of transduced cells (scored as positive when showing homogenous cytoplasmic and nucleolar fluorescence) in cells treated with linear (e) or cyclic (i) constructs. Data presented as mean±standard deviation. BF=Brightfield image. Scale bar: 20 μm.

FIG. 3. Continuous live cell confocal microscopy of trimers in Hela cells. (a) Time course uptake of 1 μM tri-Tat A; and (b) 1 μM tri-cTat B. (c, d) Cross-sectional profile plot through a representative cell treated with 1 μM tri-Tat A (c) and 1 μM tri-cTat B (d); surface plot (middle) composed of cross-sectional profiles from 1 min to 15 min (y-axis: fluorescence intensity (AU); x-axis: distance along the cross section (μm); z-axis: time (min); colour added for clarity to denote y-axis values—dark blue 0-199 AU, orange—200-399 AU, grey—400-599 AU, yellow—600-799 AU, light blue >800 AU). Tri-Tat A fluorescence moves from the plasma membrane inwards (c), while tri-cTat B fluorescence moves from a focal point in the cell outwards (d). (e-h) Time course analysis of cross sectional profiles (1-15 min) through 6 cells treated with tri-Tat A (solid lines) and tri-cTat B (dotted lines), where regions of interest (ROIs) corresponding to the membrane (e), cytosol (f), nucleus (g), and nucleoli (h) are defined and average fluorescence intensity reported. Tri-cTat B shows significantly different membrane association and uptake kinetics into cellular compartments compared to tri-Tat A. Data presented as mean±standard deviation. Scale bar: 20 μm.

FIG. 4. Membrane porosity following treatment with Tat-trimer. (a, b) Addition of 40 μM propidium iodide (PI) 20 min after addition of 1 μM trimer; image at 30 min after start of experiment. Cells treated with tri-Tat A (a) co-stain with PI; cells treated with tri-cTat B (b) are PI negative. (c) Average fluorescence intensity of PI per cell, 45 min after the start of the experiment. Cells treated with tri-Tat A show significantly higher PI uptake, indicative of pore formation. (d) Cells treated with tri-Tat A (solid line) or tri-cTat B (dotted line) for 60 min and metabolic activity as an indicator of cell viability assessed using MTT assay after 1 h, 2 h, 4 h, 3 days. Data presented as mean±standard deviation. Scale bar: 20 μm.

FIG. 5. Co-delivery of antibodies and antibody fragments in live Hela cells using tri-cTat B. (a, b) Post-wash live cell confocal microscopy images of Hela cells treated with 500 nM mouse Fab fragment AF647 conjugate (Fab-AF647) and 1 μM tri-cTat B for 30 min (a) or 166 nM mouse IgG AF647 conjugate (IgG-AF647) and 1 μM tri-cTat B for 45 min (b) show homogenous distribution of Fab in cytosol and nucleus of cells with green nucleoli staining typical of tri-cTat B delivery; (c) Continuous live-cell confocal microscopy of trimers in Hela cells treated with 500 nM Fab-AF647 (red) and 1 μM tri-cTat B (green); (d) quantification of the percentage of cells transduced with cargo (IgG-AF647/Fab-AF647-scored as positive when showing homogenous cytoplasmic and nucleolar fluorescence). Data presented as mean±standard deviation. Scale bar: 20 μm.

FIG. 6. Co-delivery of functional antibodies and antibody fragments in live Hela cells. HeLa cells were transfected with actin-RFP (a, b) or histone-RFP (c). 90 min post-wash live cell confocal microscopy images of cells treated with (a) 500 nM anti-β-actin mouse Fab AF647 conjugate (β-actin-Fab-AF647) and 1 μM tri-cTat B for 30 min show co-localization of Fab (red) with actin stress filaments (orange); (b) 166 nM anti-β-actin mouse IgG2b AF647 conjugate (β-actin-IgG-AF647) and 1 μM tri-cTat B for 30 min show co-localization of antibody with actin stress filaments; (c) 166 nM anti-RFP mouse IgG1 AF647 conjugate (RFP-IgG-AF647) and 1 μM tri-cTat B for 30 min show co-localization of antibody with RFP fused to histone in the nucleus. G=green channel (tri-cTat B); O=orange channel (RFP fusion protein); R=red channel (AF647); Co-localization panel: co-localized pixels are shown as a mask of yellow pixels of constant intensity and all results shown present a significant correlation and are co-localized; BF=brightfield; scale bar: 20 μm.

FIG. 7. UV absorbance spectra of monomers, trimers and AF488 alone.

FIG. 8. Fluorescence excitation/emission spectra of monomers, trimers, and AF488 alone.

FIG. 9. A: Live-cell confocal microscopy of live HeLa and CHO cells treated with 10 μM mono-Tat (18) for 60 min incubation; imaging post-wash. 10 μM mono-Tat is taken up only into endosomes of HeLa and CHO cells. Scale bar: 20 μm. B: Live-cell confocal microscopy of live HeLa and CHO cells treated with 10 μM mono-cTat (19) for 60 min incubation; imaging post-wash. 10 μM mono-cTat is taken up only into endosomes of HeLa and CHO cells. Scale bar: 20 μm.

FIG. 10. A: Live-cell confocal microscopy of live Hela cells transfected with rab5a-RFP and treated with 1 μM tri-Tat A (8) for 15 min; imaging pre-wash. Images from left to right represent—green channel, red channel, composite green/red channels, co-localization analysis (co-localized pixels in yellow), composite green/red/bright field channels. In cells transfected with rab5a-RFP, RFP and focal AF488 signal co-localizes, indicating that the vesicles tri-cTat B is confined are early endosomes. Results of co-localization analysis: R=0.264; M1=0.1878; M2=0.4714. Scale bar: 20 μm. B: Live-cell confocal microscopy of live Hela cells transfected with rab5a-RFP and treated with 1 μM tri-Tat B (16) for 15 min; imaging pre-wash. Images from left to right represent—green channel, red channel, composite green/red channels, co-localization analysis (co-localized pixels in yellow), composite green/red/bright field channels. In cells transfected with rab5a-RFP, RFP and focal AF488 signal co-localizes, indicating that the vesicles tri-cTat B is confined are early endosomes. Results of co-localization analysis: R=0.242; M1=0.5105; M2=0.5191. Scale bar: 20 μm.

FIG. 11. A: Live-cell confocal microscopy of live HeLa cells transfected with rab5a-RFP and treated with 1 μM tri-cTat B (17) for 15 min; imaging pre-wash. Images from left to right represent—green channel, red channel, composite green/red channels, co-localization analysis (co-localized pixels in yellow), composite green/red/bright field channels. In cells transfected with rab5a-RFP, RFP and focal AF488 signal co-localizes, indicating that the vesicles tri-cTat B is confined are early endosomes. Results of co-localization analysis: R=0.474; M1=0.7127; M2=0.6757. Scale bar: 20 μm. B: Time course of rab5a association with tri-cTat B vesicles. Live-cell confocal microscopy of live Hela cells transfected with rab5a-RFP and treated with 1 μM tri-cTat B (17); imaging pre-wash. Images from left to right: 3 min, 5 min, 7 min, 9 min, 11 min, 13 min; top row-composite green/red channels; middle row-red channel; bottom row-green channel. rab5a-RFP associates with tri-cTat B vesicles before release of the trimer into the cytosol, suggesting that tri-cTat B escapes from the early endosome. Scale bar: 20 μm.

FIG. 12. Live-cell confocal microscopy of 1 μM tri-Tat A in live Hela cells, addition of 0.5 μg propidium iodide (PI) 20 min after addition of tri-Tat A; image acquired at 30 min after start of experiment. Red channel (left), green channel (middle), and composite red/green/bright field channels (right). Cells that show uptake of both tri-Tat A and PI are marked with a cross (+); cells that show uptake of tri-Tat A only are marked with an asterisk (*). Only cells with a high level of tri-Tat in the cytosol show uptake of PI. Scale bar: 20 μm.

FIG. 13. Live-cell confocal microscopy of Fab-AF647 and 1 μM tri-cTat B in live HeLa; images acquired at 30 min after start of experiment. Red channel (left), green channel (middle), and composite red/green/bright field channels (right). Cells that show cytosolic and nuclear accumulation of both Fab-AF647 and tri-cTat B are marked with an asterisk (*); cells that show uptake of tri-cTat B only are marked with a cross (+). Accumulation of tri-cTat B and Fab are always concurrent, but some cells show accumulation of tri-cTat B without release of Fab. Scale bar: 20 μm.

FIG. 14. A; Live-cell confocal microscopy of IgG-AF647 in live Hela cells; top row: addition of 1 μM tri-cTat B; middle row: addition of 1 μM mono-cTat; bottom row: antibody only. Images acquired after 45 min of incubation, post-wash. Red channel (left), green channel (middle), and composite red/green/bright field channels (right). Cells treated with antibody only or with antibody and mono-Tat show no uptake or cytosolic accumulation of antibody; tri-cTat B is necessary for the delivery of antibody into the cytosol. Scale bar: 20 μm. B: Live-cell confocal microscopy of Fab-AF647 in live Hela cells; top row: addition of 1 μM tri-cTat B; middle row: addition of 1 μM mono-cTat; bottom row: antibody fragment only. Images acquired after 30 min of incubation, post-wash. Red channel (left), green channel (middle), and composite red/green/bright field channels (right). Cells treated with antibody fragment only or with fragment and mono-Tat show no uptake or cytosolic accumulation of antibody fragment; tri-cTat B is necessary for the delivery of antibody fragment into the cytosol. Scale bar: 20 μm.

FIG. 15. Live-cell confocal microscopy of live HeLa treated with 1 μMm (top row) and 2 μMm (middle row) tri-cTat B (17) in serum supplemented media for 60 min; imaging post-wash. Quantification of average fluorescence intensity per cell in cells treated with 1 μMm and 2 μMm tri-cTat B (17) in the presence of serum (bottom left). Quantification of the percentage of transduced cells (scored as positive when showing homogenous cytoplasmic and nucleolar fluorescence) in cells treated with 1 Mm and 2 μMm tri-cTat B (17) in the presence of serum (bottom right). Data presented as mean±standard deviation. BF=Brightfield image. Scale bar: 20 μm.

FIG. 16. Live-cell confocal microscopy of live HeLa treated with 1 μM tri-cTat B (17) and 1 μM recombinant red fluorescence protein (RFP) (top row)/2 μM recombinant RFP (second row) for 30 min in serum free media and imaged 30 min thereafter. Treatment with 1 μM recombinant RFP only (third row) and 1 μM tri-cTat B only (bottom row). Left image: red (RFP) channel; middle image: green (tri-cTat B) channel; right image: overlay of brightfield, red and green channels. Scale bar: 20 μm.

FIG. 17. Time course of cargo association with tri-cTat B (17) by live-cell confocal microscopy of live HeLa treated with: (A) 1 μM tri-cTat B and 1 μM recombinant red fluorescence protein (RFP); and (B) 1 μM tri-cTat B and 166 nM IgG antibody-AF647. Top row of each set of images: red channel (RFP/IgG-AF647); middle row: green channel (tri-cTat B); bottom row: red/green overlay. Leftmost (0 min) images are taken with cargo on the cells, but prior to the addition of tri-cTat B; 1, 2, 3, 4, 5 min images are taken at denoted time points after the addition of tri-cTat B (without washing). RFP cargo does not associate with tri-cTat B and remains entirely homogenous in extra cellular distribution, no membrane accumulation or co-localization into endosomes is observed. IgG-AF647 (while displaying homogenous extracellular distribution prior to the addition of trimer) immediately associates with tri-cTat B at the plasma membrane of cells and subsequently co-localizes into endosomes. Some aggregation of IgG in the extracellular media is also observed. Scale bar: 20 μm.

FIG. 18. Immobilized recombinant RFP protein on membrane and incubated with anti-RFP-IgG-AF647 (a) and anti-RFP-Fab-AF647 (b). ChemiDoc Imaging System (Bio-Rad) used to detect AF647 signal; IgG treated membrane exposed for 0.5 seconds; Fab treated membrane exposed for 1.8 seconds. Molecular weight marker shown on the right-hand side. Fab fragment does not retain affinity for recombinant RFP in comparison to IgG.

FIG. 19. Co-delivery of functional antibodies and antibody fragments in live Hela cells. HeLa cells were transfected with actin-RFP (A-C) or histone-RFP (D). 90 min post-wash live cell confocal microscopy images of cells treated with (A, B) 500 nM anti-B-actin mouse Fab AF647 conjugate (β-actin-Fab-AF647) and 1 μM tri-cTat B for 30 min; (C) 166 nM anti-β-actin mouse IgG2b AF647 conjugate (β-actin-IgG-AF647) and 1 μM tri-cTat B for 30 min; (D) 166 nM anti-RFP mouse IgG1 AF647 conjugate (RFP-IgG-AF647) and 1 μM tri-cTat B for 30 min. (A) Low abundance of endosomal signal and homogenous background signal throughout the cell result in similar Costes thresholding compared to ROI analysis (main manuscript). (B) Strong endosomal signal and heterogeneous background signal throughout the cell result in differences in Costes thresholding compared to subsections of the cell with more homogenous background (main manuscript). (C) Heterogeneity and large endosomal signal throughout cell, appropriate thresholds are difficult to establish. (D) Coates thresholding of the whole cell differs from subsection due spatial inhomogeneity near the nucleus and trafficking of antibody to nucleoli. G=green channel (tri-cTat B); O=orange channel (RFP fusion protein); R=red channel (AF647); Co-localization panel: co-localized pixels are shown as a mask of yellow pixels of constant intensity and all results shown present a significant correlation and are co-localized; BF=brightfield; scale bar: 20 μm.

FIG. 20. Proximity ligation assay (PLA). The PLA were performed in Hela cells using an H2B-AF488 antibody and a histone H2A.Z antibody. H2B-AF488 (166 nM) was delivered into cells using cTat3-alkyne (2 μM, 1 h incubation at 37° C., 5% CO2). Negative controls were cells incubated with either H2B-AF488 alone, or H2A.Z antibodies alone following fixation and permeabilization of the cells. (a) Confocal microscopy images showing Hoechst 33342 stained nuclei (blue), internalised H2B-AF488 (green), PLA signals (red) and an overlay of the fluorescent channels. (b) The number of nuclear PLA signals was quantified using CellProfiler and represented using Origin. Data presented as mean±SD. One way ANOVA and Tukey's test was employed in statistical analysis. **p<0.01. Scale bars: 20 μm.

FIG. 21. Quantification of the percentage of transfected cells (scored as positive when showing homogenous cytoplasmic and nucleolar fluorescence) in Hela cells treated with linear tri-Tat constructs having 3, 5, 7 and 9 PEG units (tri-Tat C, D, E and F; 20, 21, 22 and 23) compared to tri-Tat A (8) and tri-cTat B (17), which have 0 and 1 PEG unit, respectively. Data presented as mean±standard deviation.

FIG. 22. Quantification of the percentage of transfected cells (scored as positive when showing homogenous cytoplasmic and nucleolar fluorescence) in Hela cells treated with cyclic tri-Tat constructs having 3, 5, 7 and 9 PEG units (tri-cTat C, D and E; 24, 25 and 26) compared to tri-Tat A (8) and tri-cTat B (17), which have 0 and 1 PEG unit, respectively. Data presented as mean±standard deviation.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Sambrook et al., Molecular Cloning: A Laboratory Manual (2d ed., Cold Spring Harbor Laboratory Press, Cold Spring Harbor, N.Y. (1989)). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

In a first aspect the invention relates to a compound comprising formula I;

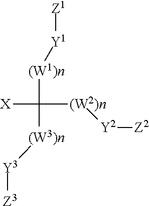

wherein;

W is an extension moiety,

X is selected from a linking moiety, a cargo moiety, a fluorophore, or a H

Y is a linking moiety,

Z is a cell penetrating peptide comprising SEQ ID NO:1 (RXXRRXRRR)

wherein n=0 to 10, and wherein Z is joined to Y via one atom of Z, or is joined to Y via at least two atoms of Z such as to form a cyclic moiety with Y.

The atom at the core of Formula I is a carbon, as such Formula I can also be represented as below.

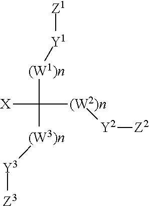

In some embodiments the cell penetrating peptide (CPP) comprises SEQ ID NO:1 RXXRRXRRR wherein amino acid X may be arginine (R), lysine (K) or glutamine (Q). The CPP may be based on the sequence of the CPP TAT. TAT is the trans-activating transcriptional activator (TAT) from human immunodeficiency virus 1 (HIV-1). In particular the CPP may be based on residues 49-57 of TAT. In an embodiment the CPP comprises SEQ ID NO:2 RKKRRQRRR. The CPP may comprise a sequence with at least 75% sequence identity to SEQ ID NO:2, or at least 77% sequence identity to SEQ ID NO:2, or at least 85% sequence identity to SEQ ID NO:2, or at least 88% sequence identity to SEQ ID NO:2. The CPP may comprise additional amino acid residues either at the N or C terminal. There may be an additional 1, 2, 3, 4, or 5 additional amino acid residues present at either the N or C terminal of the CPP. These additional amino acid residues may reflect the amino acids that are present in the sequence of TAT e.g. residues 44-62 of TAT. For example, the CPP may comprise SEQ ID NO:3 GRKKRRQRRRPQ. The additional amino acids at the N or C terminal may have a positive net charge.

TABLE 1
Sequences of cell penetrating peptides

| SEQ ID NO. | Length | Molecule Type | Residues |
|---|---|---|---|
| SEQ ID NO. 1 | 9 | amino acid | RXXRRXRRR |
| | | (synthetic construct) | wherein X can be any naturally |
| | | | occurring organism |
| SEQ ID NO: 2 | 9 | amino acid | RKKRRQRRR |
| | | (synthetic construct) | |
| SEQ ID NO: 3 | 12 | amino acid | GRKKRRQRRRPQ |
| | | (synthetic construct) | |

As used herein the term “% sequence identity” refers to the percentage of amino acid residues in a polypeptide sequence that are identical to the amino acid sequence of a specified polypeptide sequence. For example, percentage identity between two protein sequences can be determined by pairwise comparison of the two sequences using the bl2seq interface at the Web site of the National Center for Biotechnology Information (NCBI), U.S. National Library of Medicine, 8600 Rockville Pike, Bethesda, MD 20894, U.S.A. The b!2seq interface permits sequence alignment using the BLAST tool described by Tatiana, A., et al., “Blast 2 Sequences—A New Tool for Comparing Protein and Nucleotide Sequences,” FEMS Microbiol, Lett. 174:247-250 (1999).

In an embodiment the CPP has a positive net charge. The positive net charge may be contributed to by the amino acid side chains in particular the presence of the guanidium groups in the arginine side chains and/or the ammonium groups in the lysine side chains.

In an embodiment the CPP is linear. Where the CPP is linear it may be attached via the C terminus or the N terminus of the CPP to the linking moiety Y, the other end is not attached to the linking moiety. The term “C terminus” as used herein refers to the end of the peptide comprising a carboxy group or a carbonyl group. The term “N terminus as used herein refers to the end of the peptide comprising an amino group or an amine group

In an embodiment, in order to form the linking moiety (Y) the linear CPP may further comprise an N-terminus modified with a hexanoyl. The CPP may then be conjugated to an azide group via the alkyne of the hexanoyl group to form the linking moiety (Y). Suitable hexanoyl compounds include but are not limited to hexanoyl chloride. Alternatively, the linear CPP may comprise an N-terminus modified with an azido-pentanoyl. The CPP may then be conjugated to an alkyne group via the azido group to form the linking moiety (Y).

Alternatively, the cell penetrating peptide may be cyclic. Where the CPP is cyclic, the cyclisation may be achieved using any known method. The cyclisation method may involve introducing an azide modified lysine (K(N₃) at the N terminus of the CPP and introducing a glutamic acid at the C terminus of the CPP, which can then undergo cyclisation between the carboxylic acid of the glutamic acid and the modified lysine. The cyclisation method may involve introducing a propagyl glycine at the N terminus of the CPP and introducing a glutamic acid at the C terminus of the CPP, which can then undergo cyclisation between the carboxylic acid of the glutamic acid and the modified glycine. In an embodiment the CPP is cyclised via a C terminal glutamic acid and an N-terminal azide-modified lysine or propagyl glycine.

In an embodiment the CPP is cyclic, in this embodiment the CPP Z may be joined to the linking moiety Y via at least two atoms of Z such as to form a cyclic moiety with Y. The attachment of Y to Z may occur at the peptide backbone of the cyclic peptide. In an embodiment the attachment of Y to Z may occur at a Cα of the peptide backbone of the cyclic peptide.

In an embodiment, the linking moiety is formed via conjugation of the CPP to the central scaffold of the compound. In order to form the linking moiety (Y) the cyclic CPP may be conjugated to an azidogroup via the alkyne group of the propagyl glycine. The cyclic CPP may be conjugated to an alkyne group via the azido group of the azide modified lysine to form the linking group (Y).

The compound of Formula I may be formed by taking a central scaffold and attaching three linear and/or cyclic CPPs. The central scaffold may comprise azide and/or alkyne functionalized groups. The central scaffold may also comprise the extension moiety. The three CPPs may comprise complementary azide and/or alkyne functionalized groups which can react to link the CPP to the scaffold. This reaction may in turn form the linking moiety. Strain promoted azide-alkyne cycloaddition reactions may be used to attach the CPP molecules to the central scaffold, the reaction may occur between a central scaffold comprising an azide or tetrazine functionalised group and an CPP molecule comprising a strained cyclo-alkyne functionalised group. Alternatively, the reaction may occur between a central scaffold comprising a strained cyclo-alkyne functionalised group and a CPP molecule comprising an azide or tetrazine functionalised group.

The scaffold may comprise a compound of Formula II;

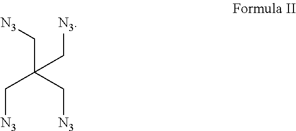 [SMILES: [N-]=[N+]=NCC(CN=[N+]=[N-])(CN=[N+]=[N-])CN=[N+]=[N-]]

The central scaffold may comprise Formula III:

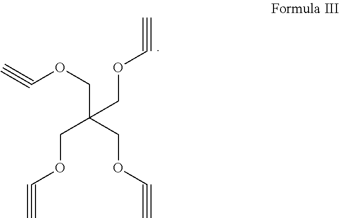 [SMILES: C#COCC(COC#C)(COC#C)COC#C]

The scaffold may comprise a compound of the following formula: C—[CH₂—(O—CH₂—CH₂—O—)ₙ-CH2—CH2—O—C—N₃]₄, wherein n is an integer from 1 to 4.

The scaffold may comprise a compound of the following formula: C—[CH₂—(O—CH₂—CH₂—O—)ₙ-CH2—CH2—O—C≡CH]₄, wherein n is an integer from 1 to 4.

The compound of Formula I comprises three CPPs, these CPPs may be in the same or different formats. For example, the three CPPs (Z¹, Z², Z³) may all be in linear format. The three CPPs (Z¹, Z², Z³) may all be in cyclic format. The CPPs (Z¹, Z², Z³) may be in a mixture of formats with some being in linear format and some being in cyclic format. For example, one CPP may be in linear format and two may be in cyclic format. Two CPPs may be in linear format and one may be in cyclic format.

In an embodiment the linking moiety may comprise a C3-C12 cycloalkyl, C3-C12 heterocycloalkyl, a C3-C12 aryl, a 3-membered to 12-membered heteroaryl, or a C3-C10 cycloalkyl, C3-C10 heterocycloalkyl, a C3-C10 aryl, a 3-membered to 10-membered heteroaryl, or C3-C8 cycloalkyl, C3-C8 heterocycloalkyl, a C3-C8 aryl, a 3-membered to 8-membered heteroaryl, or a C3-C6 cycloalkyl, C3-C6 heterocycloalkyl, a C3-C6 aryl, a 3-membered to 6-membered heteroaryl, or C3-C5 cycloalkyl, C1-C5 heterocycloalkyl, a C3-C5 aryl, a 3-membered to 5-membered heteroaryl, or a C3-C4 cycloalkyl, C1-C4 heterocycloalkyl, a C3-C4 aryl, a 3-membered to 4-membered heteroaryl. The linking moiety may comprise a 4-membered to 6-membered heteroaryl, preferably the linking moiety comprises a 5 membered heteroaryl.

The linking moiety may comprise one or more substituent groups. The cycloalkyl, heterocycloalkyl, aryl, or heteroarylgroup of the linking moiety may be optionally substituted with one or more substituents selected from C1-C6 alkyl, C1-C6 alkenyl, C1-C6 alkoxy, C1-C6 haloalkyl, preferably C1-C4 alkyl, C1-C4 alkenyl, C1-C4 alkoxy, C1-C4 haloalkyl. The linking moiety may comprise an optionally substituted cycloalkyl, an optionally substituted heterocycloalkyl, an optionally substituted aryl or an optionally substituted heteroaryl, wherein each cycloalkyl, heterocycloalkyl, aryl or heteroaryl is optionally substituted with one or more substituents, selected from C1-C6 alkyl, C1-C6 carbonylalkyl, C1-C6 hydroxyalkyl, C1-C6 alkoxy or C1-C6 alkyl aminocarbonyl, preferably a C1-C4 alkyl, C1-C4 carbonylalkyl, C1-C4 hydroxyalkyl, C1-C4 alkoxy or C1-C4 alkyl aminocarbonyl. In an embodiment 1 to 3 substituents may be present. One of the substituent groups may link the linking group to the CPP.

The linking group may comprise a monocyclic or polycyclic moiety for example a bicyclic or tricyclic moiety. The linking group may comprise monocyclic, bicyclic, tricyclic or polycyclic cycloalkyl, or a monocyclic, bicyclic, tricyclic or polycyclic heterocycloalkyl, or a monocyclic, bicyclic, tricyclic or polycyclic aryl, or a monocyclic, bicyclic, tricyclic or polycyclic heteroaryl.

The term “alkyl” refers to an optionally substituted saturated aliphatic branched or straight-chain monovalent hydrocarbon radical having the specified number of carbon atoms.

The term “aryl” or “aromatic” refers to an aromatic monocyclic or polycyclic (e.g. bicyclic or tricyclic) carbocyclic ring system. In one embodiment, “aryl” is a 3-12 membered monocylic or bicyclic system. Aryl systems include, but not limited to, phenyl, naphthyl, fluorenyl, indenyl, azulenyl, and anthracenyl. In an embodiment, “aryl” is phenyl.

The term “cycloalkyl” refers to a saturated aliphatic cyclic hydrocarbon ring. Thus, “C3-C10 cycloalkyl” means a hydrocarbon radical of a 3-10 membered saturated aliphatic cyclic hydrocarbon ring. A C3-C10 cycloalkyl includes, but is not limited to cyclopropyl, cyclobutyl, cyclopentyl, cyclohexyl, cycloheptyl, cyclooctyl, cyclononyl, cyclodecyl.

The term “hetero” refers to the replacement of at least one carbon atom member in a ring system with at least one heteroatom selected from N, S, and O. “Hetero” also refers to the replacement of at least one carbon atom member in an acyclic system. A hetero ring system or a hetero acyclic system may have 1, 2, 3 or 4 carbon atom members replaced by a heteroatom.

The term “heterocycloalkyl” refers to monocyclic or polycyclic ring comprising carbon and hydrogen atoms and at least one heteroatom, preferably, selected from nitrogen, oxygen, and sulfur. The heterocycloalkyl may comprise one or more hetero atoms for example 1 to 10, 1 to 8, 1 to 6, 1 to 4 or 1 to 2 hetero atoms. Heterocycloalkyl groups may include, but are not limited to aziridinyl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl, and pyranyl. A heterocycloalkyl group may be unsubstituted or substituted with one or more suitable substituents. The heterocycloalkyl group may be a monocyclic, bicyclic, tricyclic or multicyclic ring.

In an embodiment the linking moiety comprises a heterocyclic group, selected from a four-membered ring, a five membered ring, or a six membered ring. The linking moiety comprises 1 to 5 hetero atoms.

The term “heterocyclic” of “heterocyclyl” refers to a cyclic 4 to 12 membered saturated or unsaturated aliphatic or aromatic ring containing 1, 2, 3, 4 or 5 heteroatoms independently selected from preferably nitrogen, oxygen, and sulfur.

The linking moiety may comprise a triazole for example a 1, 2, 3 triazole or a 1, 2, 4 triazole.

In an embodiment the extension moiety is a hydrophilic polymer. The hydrophilic polymer may be selected from (—CH2—)ₙ, (—CH2—O—CH2—)ₙ and or PEG alternatives. A suitable PEG alternative may be selected from poly(glycerols) (PGs), poly(oxazolines) (POX), poly(hydroxypropyl

methacrylate) (PHPMA), poly(2-hydroxyethyl methacrylate) (PHEMA) [71], poly(N-(2-hydroxypropyl) methacrylamide) (HPMA), poly(vinylpyrrolidone) (PVP), poly(N,N-dimethyl acrylamide) (PDMA), poly(N-acryloylmorpholine) (PAcM), hyaluronic acid (HA), Heparin, or polysialic acid (PSA). In a preferred embodiment the extensions moiety is not charged, zwitterionic or strongly lipophilic such as aromatic groups and alkyl chains.

In an embodiment the extension moiety may be longer or shorter, for example n=0 to 9, or 0 to 8, or 0 to 7, or 0 to 6, or 0 to 5, or 0 to 4, or 0 to 3 or 0 to 2 or 0 to 1. In an embodiment n=5, or 4, or 3, or 2, or 1.

In an embodiment, the present invention does not encompass trimers of tat presented on a scaffold of amino acids. In particular, it does not encompass trimers of tat presented on a scaffold of lysine residues.

In a preferred embodiment the compound of Formula I comprises or consists of Formula IV, Formula V, Formula VI, or Formula VII.

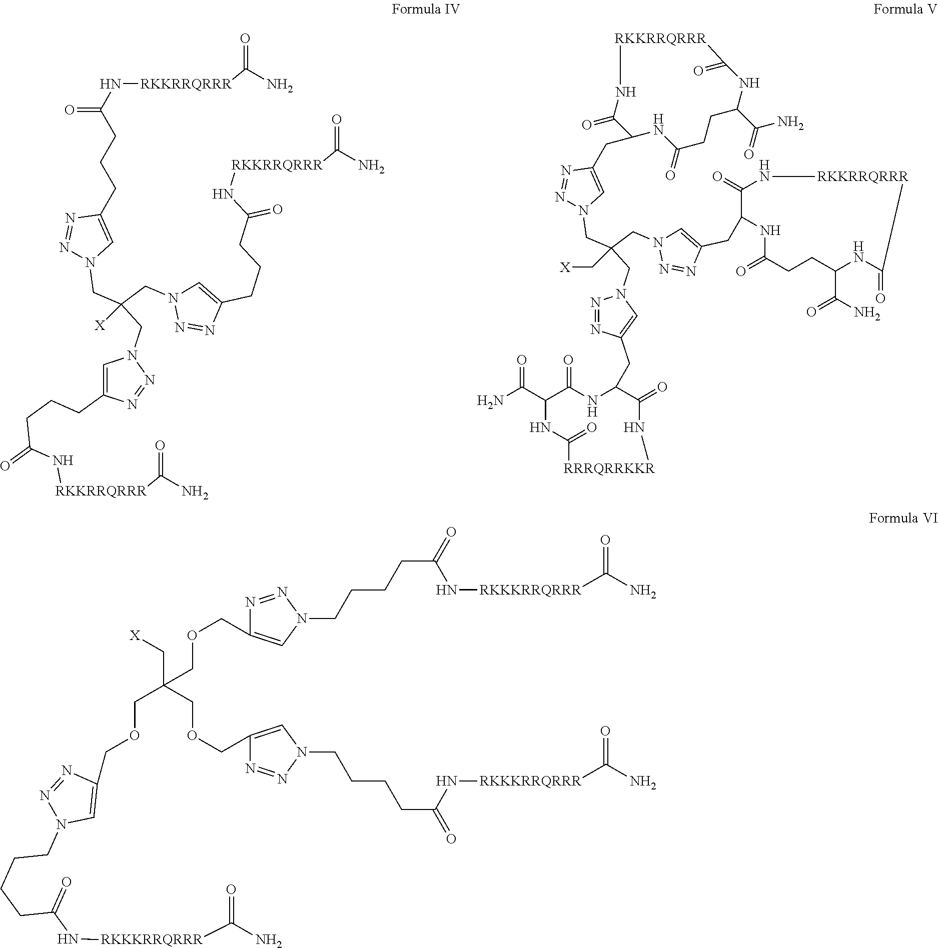

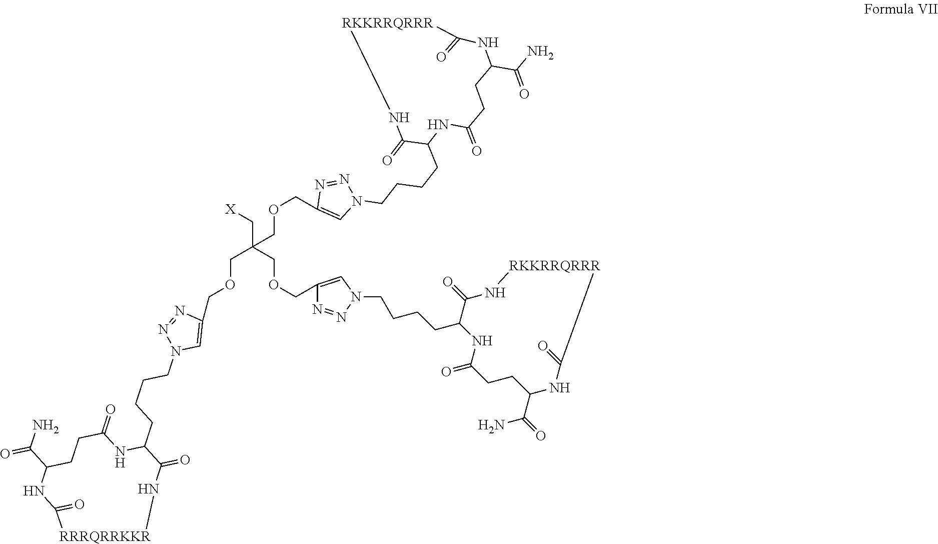

In Formula IV, V, VI and VII, X is selected from a linking moiety, a cargo moiety, a fluorophore, or a H.

In an embodiment the cargo moiety is selected from a fluorophore, a small molecule therapeutic, a therapeutic peptide, a therapeutic moiety, a protein, an antibody, an antigen-binding fragment, a single chain variable fragment, a single domain antibody, antibody fragment, or an oligonucleotide.

The term “antibody”, broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

An antibody fragment as used herein is a portion of an antibody, for example as F (ab′)2, Fab, Fv, sFv and the like. Functional fragments of a full length antibody retain the target specificity of a full length antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs. scFv fragments (˜25 kDa) consist of the two variable domains, VH and VL. Naturally, VH and V|_ domain are non-covalently associated via hydrophobic interaction and tend to dissociate. However, stable fragments can be engineered by linking the domains with a hydrophilic flexible linker to create a single chain Fv (scFv). The smallest antigen binding fragment is the single variable fragment, namely the VH or VL domain. Binding to a light chain/heavy chain partner respectively is not required for target binding. Such fragments are used in single domain antibodies. A single domain antibody (−12 to 15 kDa) therefore has either the VH or VL domain. In one embodiment, the antibody fragment is a Fab.

The antibody or antibody fragment may be conjugated to a payload for intracellular delivery of a toxic compound. In another embodiment, the antibody or antibody fragment may be linked to a radionuclide for radiotherapy.

Antibodies of interest include those that target intracellular targets.

Suitable fluorophores that may be cargo moieties include AlexaFluor488, Alexa Fluor 555, Alexa Fluor 594, Oregon Green, Rhodamine, fluorescein isothiocyanate (FITC), tetramethylrhodamine (TRITC), cyanine fluorophores and sulfo-cyanine fluorophores.

The term “therapeutic peptide” refers to a short chain of amino acids. The therapeutic peptide may be linear without secondary or tertiary structure. The therapeutic peptide may comprise approximately 5 to 50 amino acids.

The term “protein” refers to a compound made up of predominantly amino acids. Suitable protein cargos proteins with inhibitory function, enzymes, fusion proteins. In particular mini-proteins such as Omomyc may be used and enzymes with endonuclease activity including Cas enzymes such as Cas9, Cas12, Cpf1 may be used.

Oligonucleotides may be suitable cargo moieties, these may be therapeutic oligonucleotides such as antisense gapmers, steric blocks, splice-switching ONs, short interfering RNAs, antagomirs (antimicroRNAs), microRNA mimics, aptamers, DNA decoys, DNAzymes.

The cargo moiety may further comprise an extension moiety and/or a linking moiety as described herein. Alternatively, the cargo moiety may comprise an extension moiety and/or a linking moiety which is different to the extension moieties W¹⁻³ or the linking moieties Y¹⁻³. For example the extension moiety and/or the linking moiety may comprise (Gly₄Ser)ₙ linkers or cysteine rich linkers, non-canonical amino acid linkers. The extension moiety and/or linking moiety may be present to attach the cargo moiety to the central scaffold.

In an aspect of the invention the compound according to Formula I is for use as a cell delivery agent for intracellular delivery. The inventors have shown that the trimeric CPPs of Formula I can be used to deliver functional biomacromolecules to the cytosol and nucleus of the cell, as such these delivery agents are extremely useful cell-based assays, cellular imaging applications, ex vivo manipulation and reprogramming of cells, and in vivo administrations of therapeutic agents, among other applications.

The compound according to Formula I can also be used as a co-delivery agent for intracellular delivery of a therapeutic moiety, wherein the co-delivery is of an antibody, antigen-binding fragment, single chain variable fragment, single domain antibody, antibody fragment, protein, peptide or an oligonucleotide into a cell. The term “co-delivery agent” refers to an agent that can be used to facilitate delivery of a molecule to a cell, wherein the molecule is not covalently linked to the co-delivery agent. Where the compound of Formula I is used as a co-delivery agent it may be preferred that X is selected from a linking moiety, a fluorophore, or a H. For co-delivery, the trimeric CPP molecule of Formula I is co-administered with an antibody, antigen-binding fragment, single chain variable fragment, single domain antibody, antibody fragment, protein, peptide or an oligonucleotide to a cell. The administration of the compound of Formula I may be separately, sequentially or simultaneously with the molecule to be delivered, for example the antibody, antigen-binding fragment, single chain variable fragment, single domain antibody, antibody fragment, or an oligonucleotide.

The compound of Formula I may be used as an in vitro diagnostic agent, a therapeutic administration agent and/or a cellular imaging agent.

An aspect of the present invention related to the compound according to Formula I, for use in therapy.

The invention also relates to a method of treatment comprising the administration of a compound according to Formula I.

The invention also relates to the use of a compound according to Formula I in the manufacture of a medicament for therapeutic application.

The therapy or method of treatment may further comprise administration of a therapeutic agent such as an antibody, antigen-binding fragment, single chain variable fragment, single domain antibody, antibody fragment, or an oligonucleotide. Administration of the further therapeutic agent may be performed separately, sequentially or simultaneously with the compound of Formula I.

The therapy may also further comprise administration of a chemotherapy agent.

An aspect of the present invention relates to a method of delivering a molecule into a cell comprising contacting a cell with the compound of Formula I. In an embodiment the molecule is a cargo moiety which is covalently linked to the compound of Formula I. In an alternative embodiment the molecule is not covalently linked to the compound of Formula I. The method of delivering a molecule into a cell may comprise delivery of a cell impermeable molecule, wherein the molecule does not enter the cell when not in the presence of the compound of Formula I. The method of delivering a molecule into a cell may comprise delivery of a molecule such as an antibody, antigen-binding fragment, single chain variable fragment, single domain antibody, antibody fragment, a small molecule therapeutic or an oligonucleotide.

In an embodiment the method of delivering a molecule into a cell comprises contacting a cell separately, sequentially or simultaneously with the compound of Formula I and the molecule to be delivered. In an embodiment the method of delivering a molecule into a cell is performed on a cell ex vivo or in vivo. The method of delivering a molecule to a cell may deliver the molecule to the cytosol or to the nucleus of the cell.

The method of delivering a molecule into a cell may comprise contacting a cell with a compound of Formula I at a concentration of between 0.1 μM and 5 μM, or between 0.5 μM and 4 μM, or between 0.5 μM and 3 μM, or between 0.5 μM and 2 μM, or between 0.5 μM and 1 μM.

An aspect of the invention relates to a method of producing a cyclic Tat peptide comprising the steps of;
incorporating an azide modified lysine residue or a propagyl glycine to the N-terminus of the Tat peptide,
incorporating a glutamic acid to the C-terminus of the Tat peptide, and performing head-to-tail cyclisation.

In an embodiment the Tat peptide used in the method of cyclisation may comprise SEQ ID NO:1 (RXXRRXRRR). Preferably the Tat peptide used in the method of cyclisation comprises SEQ ID NO:2 (RKKRRQRRR), or a sequence with at least 75% sequence identity to SEQ ID NO:2, or at least 77% sequence identity to SEQ ID NO:2, or at least 85% sequence identity to SEQ ID NO:2, or at least 88% sequence identity to SEQ ID NO:2. In an embodiment the C-terminus of the Tat peptide is aminated.

In an aspect the invention relates to a method of delivering a cell impermeable molecule to the cytosol and/or nucleus of a cell, comprising contacting a cell with the compound of Formula 1 and a cell impermeable molecule.

In an embodiment the cell-impermeable molecule may be an antibody, antigen-binding fragment, single chain variable fragment, single domain antibody, antibody fragment, a small molecule therapeutic, protein, peptide or an oligonucleotide.

In an aspect the invention relates to a method of producing a trimeric cell penetrating peptide, comprising reacting a scaffold comprising a compound of Formula II or Formula III, with three CPP molecules comprising an alkyne functionalised group or an azide functionalised group.

The CPP molecules used in the method of producing a trimeric cell penetrating peptide may be a Tat peptide. The peptide may comprise SEQ ID NO:1 (RXXRRXRRR). Preferably the Tat peptide used in the method of producing a trimeric cell penetrating peptide comprises SEQ ID NO:2 (RKKRRQRRR), or a sequence with at least 75% sequence identity to SEQ ID NO:2, or at least 77% sequence identity to SEQ ID NO:2, or at least 85% sequence identity to SEQ ID NO:2, or at least 88% sequence identity to SEQ ID NO:2.

The CPP which is attached to the scaffold comprising Formula II or Formula III may be cyclic. Where the CPP is cyclic, the cyclisation may be achieved using any known method. The cyclisation method may involve introducing an azide modified lysine (K(N₃) at the N terminus of the CPP and introducing a glutamic acid at the C terminus of the CPP, which can then undergo head to tail cyclisation to the N terminus. The cyclisation method may involve introducing a propagyl glycine at the N terminus of the CPP and introducing a glutamic acid at the C terminus of the CPP, which can then undergo head to tail cyclisation to the N terminus.

In an embodiment the scaffold may comprise azide and/or alkyne functionalized groups. The central scaffold may further comprise an extension moiety. The three CPPs may comprise an azide and/or alkyne functionalized groups which can react to link the CPP to the scaffold. This reaction may in turn form a linking moiety. In an embodiment the scaffold comprises Formula II and each CPP comprises an alkyne functionalised group. In an embodiment the scaffold comprises Formula III and each CPP comprises an azide functionalised group. Strain promoted azide-alkyne cycloaddition reactions may be used to attach the CPP molecules to the central scaffold, the reaction may occur between a central scaffold comprising an azide or tetrazine functionalised group and an CPP molecule comprising a strained cyclo-alkyne functionalised group. Alternatively the reaction may occur between a central scaffold comprising a strained cyclo-alkyne functionalised group and a CPP molecule comprising an azide or tetrazine functionalised group.

In an embodiment the CPP is cyclised via a C terminal glutamic acid and a N-terminal azide-modified lysine or propagyl glycine.

The invention also relates to an immunuconjugate comprising a compound as described herein and a therapeutic moiety, for example an antibody or fragment thereof.

The invention also relates to a pharmaceutical composition comprising a compound according or an immunuconjugate described herein.

The invention also relates to a kit comprising a compound, an immunuconjugate or a pharmaceutical composition as described herein.

The invention also relates to an vivo or in vitro method for enhancing antibody or antibody fragment penetration into the cytosol of a target cell.

The terms “comprise,” and “include” are open-ended linking verbs. For example, any method that “comprises,” or “includes” one or more steps is not limited to possessing only those one or more steps and also covers other unlisted, steps. As an alternative to or in addition to “comprising,” any embodiment herein can recite “consisting of.” The transitional phrase “consisting of excludes any element, step, or ingredient not specified in the claim.

The invention is further defined in the following numbered embodiments:
1. A compound comprising formula I;

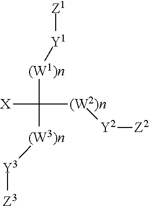

wherein;

W is an extension moiety,

X is selected from a cargo moiety, or H

Y is a linking moiety,

Z is a cell penetrating peptide comprising SEQ ID NO:1 (RXXRRXRRR).

wherein n=0 to 10, and wherein Z is joined to Y via one atom of Z, or is joined to Y via at least two atoms of Z such as to form a cyclic moiety with Y.
2. A compound according to embodiment 1, wherein the cell penetrating peptide comprises SEQ ID NO:2 (RKKRRQRRR).
3. A compound according to embodiment 1 or 2, wherein the cell penetrating peptide is linear.
4. A compound according to embodiment 1 or 2, wherein the cell penetrating peptide is cyclic.
5. A compound according to embodiment 4, wherein the cell penetrating peptide is cyclised via a C terminal glutamic acid and a N-terminal azide-modified lysine (K(N)₃) or propagyl glycine (Pra).
6. A compound according to any preceding embodiment, wherein n=0 to 5.
7. A compound according to any preceding embodiment, wherein the extension moiety is a hydrophilic polymer.
8. A compound according to any preceding embodiment, wherein the extension moiety is selected from (—CH₂—), (—CH₂—O—CH₂—), poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), poly(N-acryloylmorpholine) (PAcM), hyaluronic acid (HA), Heparin, or polysialic acid (PSA).
9. A compound according to embodiment 8, wherein the extension moiety is selected from (—CH₂—) or (—CH₂—O—CH₂—).
10. A compound according to embodiment 9, wherein the extension moiety is (—CH₂—) and n=1 to 5.
11. A compound according to embodiment 9, wherein the extension moiety is (—CH₂—O—CH₂—) and n=0 to 5.
12. A compound according to any preceding embodiment, wherein the linking moiety comprises a C3-C12 cycloalkyl, C3-C12 heterocycloalkyl, a C3-C12 aryl, a 3-membered to 12-membered heteroaryl.
13. A compound according to any preceding embodiment, wherein the linking moiety comprises an optionally substituted cycloalkyl, an optionally substituted heterocycloalkyl an optionally substituted aryl or an optionally substituted heteroaryl, wherein each cycloalkyl, heterocycloalkyl, aryl or heteroaryl is optionally substituted with one or more groups, selected from C1-C6 alkyl, C1-C6 carbonylalkyl, C1-C6 hydroxyalkyl, C1-C6 alkoxy or C1-C6 alkyl aminocarbonyl.
14. A compound according to any preceding embodiment, wherein the linking moiety comprises a heterocyclic group, selected from a four-membered ring, a five membered ring, or a six membered ring.
15. A compound according to any of embodiments 12 to 14, wherein the linking moiety comprises 1 to 5 hetero atoms, preferably 1 to 3 hetero atoms.
16. The compound according to any preceding embodiment, wherein X comprises a cargo moiety wherein the cargo moiety is selected from a fluorophore, a small molecule therapeutic, a therapeutic peptide, a therapeutic protein, an antibody, an antigen-binding fragment, a single chain variable fragment, a single domain antibody, antibody fragment, or an oligonucleotide.
17. The compound according to embodiment 16, wherein the cargo moiety further comprises an extension moiety and/or a linking moiety.
18. The compound according to any preceding embodiment, for use as a cell delivery agent.
19. The compound according to any preceding embodiment, for use as a co-delivery agent, wherein the co-delivery is of an antibody or fragment thereof or an oligonucleotide into a cell.
20. The compound according to any preceding embodiment, for use as an in vitro diagnostic agent.
21. The compound according to any preceding embodiment, for use in therapy.
22. A method of delivering a molecule into the cytosol of a cell comprising contacting a cell with the compound of any of claims 1 to 16.
23. The method of embodiment 22, wherein the molecule is covalently linked to the compound.
24. The method of embodiment 22, wherein the molecule is not covalently linked to the compound.
25. The method of any of embodiments 22 to 24, wherein the method is performed on a cell in vitro, ex vivo or in vivo.
26. A method of producing a cyclic Tat peptide comprising the steps of;
   incorporating an azide modified lysine residue or a propagyl glycine to the N-terminus of the Tat peptide,
   incorporating a glutamic acid to the C-terminus of the Tat peptide, and performing head-to-tail cyclisation.
27. The method according to embodiment 26, wherein the Tat peptide comprises SEQ ID NO:1 (RXXRRXRRR).
28. The method according to embodiment 26 or 27, wherein the Tat peptide comprises SEQ ID NO:2 (RKKRRQRRR).
29. An immunoconjugate comprising a compound according to any of embodiment 1 to 16 and a therapeutic moiety.
30. The immunoconjugate of embodiment 29 wherein the therapeutic moiety is an antibody or fragment thereof.
31. A pharmaceutical composition comprising a compound according to any of embodiments 1 to 16 or an immunoconjugate of embodiments 29 to 30.
32. A kit comprising a compound according to any of embodiments 1 to 16 or an immunoconjugate of embodiments 29 to 30.

### EXAMPLES

The invention is further described in the following non-limiting examples.

### Tat Trimers A and B

### Example 1. Synthesis of Tat Trimers A and B

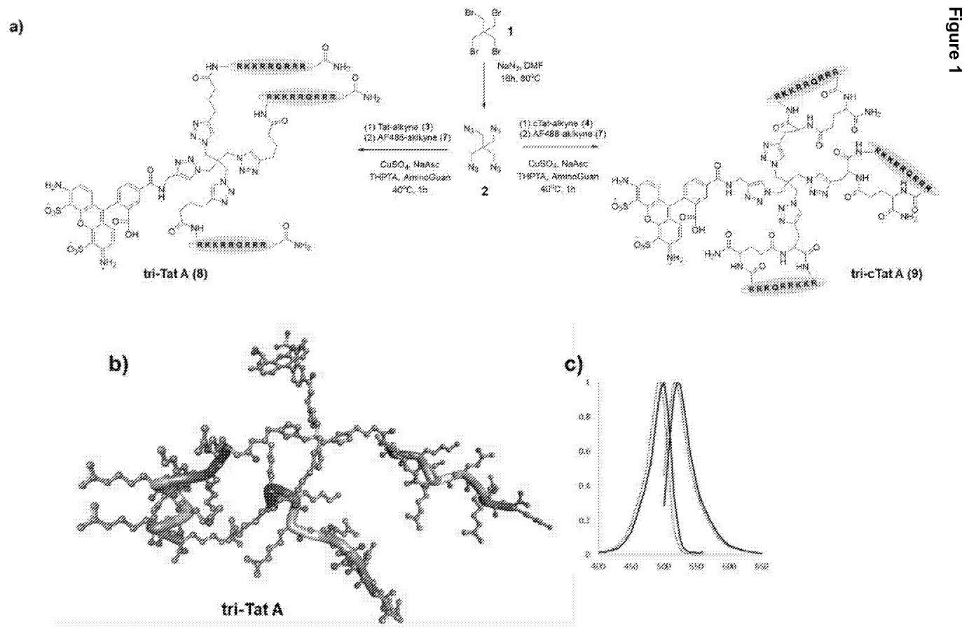

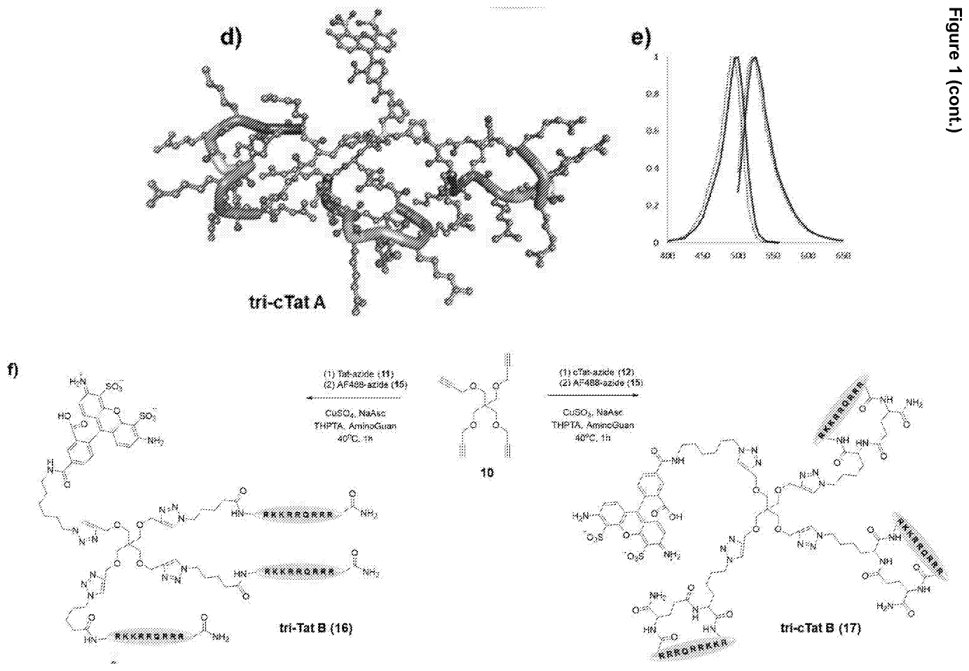

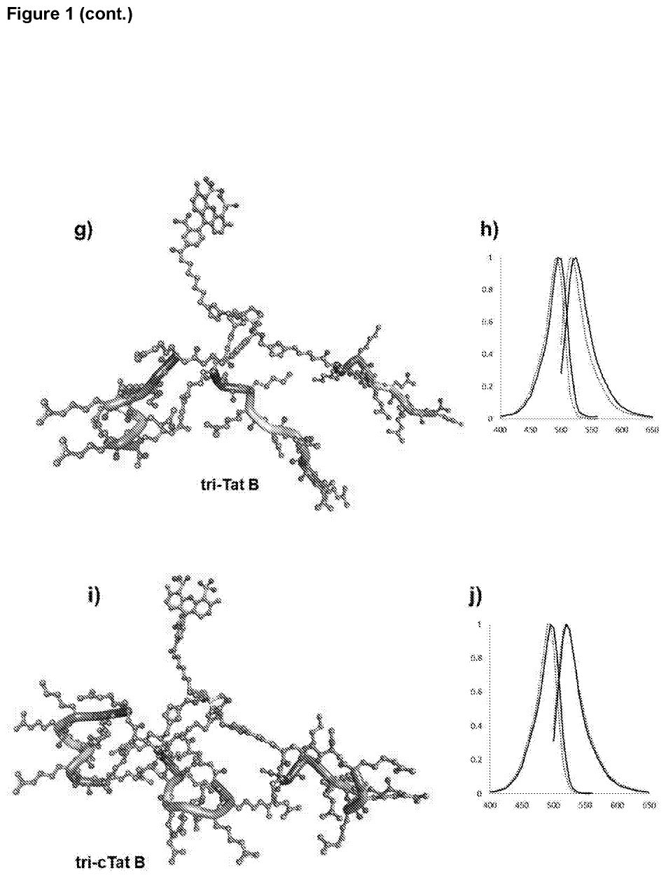

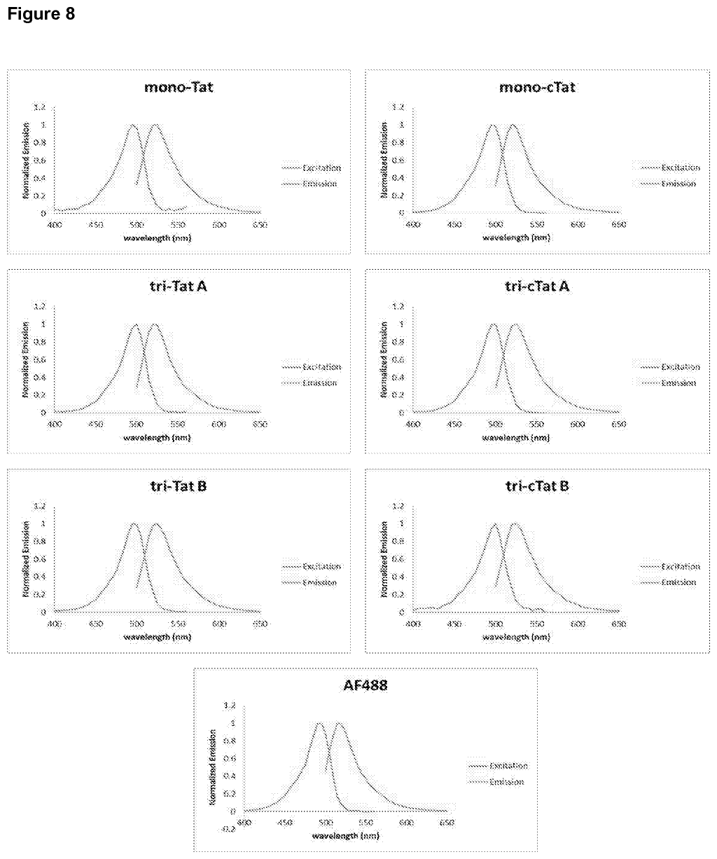

Four Tat trimers were designed using two different scaffolds furnished with either linear or cyclic Tat (cTat) peptide. These were chosen to determine (1) whether Tat trimers are more effective than monomers, (2) whether scaffold geometry affects the efficacy of Tat trimers, and (3) whether trimers based on cyclic sequences behave differently from trimers based on linear peptide. Tat-trimers were synthesized by copper catalysed 2+3 cyclo-addition between azide and alkyne functionalized components. The synthesis of trimers tri-Tat A and tri-cTat A based on azide functionalized scaffold A (2—FIG. 1a) is shown in FIG. 1a; the synthesis of trimers tri-Tat B and tri-cTat B is based on an alkyne functionalized scaffold B (10—FIG. 1f) Linear Tat (49-57) peptide (RKKRRQRRR), modified with a hexynoyl (alkyne) (3) or an azido-pentanoyl (azide) (11) at the N-terminus was used to synthesize tri-Tat A (8) and tri-Tat B (16), as well as monomeric control mono-Tat (18). Cyclic Tat was custom designed to match the linear sequence; azide/alkyne functionality was added by including either an azide modified lysine (K(N₃)) residue or propargyl glycine (Pra) at the N-terminus. Peptides were cyclized by addition of glutamic acid to the C-terminus for head to tail cyclisation to the N-terminus to generate cyclic Pra-RKKRRQRRRG (4) and K(N₃) RKKRRQRRRG (12). These peptides were used to synthesize cyclic trimers tri-cTat A (9) and tri-cTat B (17), as well as monomeric cyclic control mono-cTat (19). Tat-trimers were labelled with AlexaFluor488 (AF488) for further study. Photophysical properties of peptide trimers were found to be similar to AF488 alone (FIGS. 1, 7 and 8). In silico generated three-dimensional representations of all four trimers are shown as ball and stick models in FIG. 1.

### Example 2. Uptake of Trimers in HeLa and CHO Cells

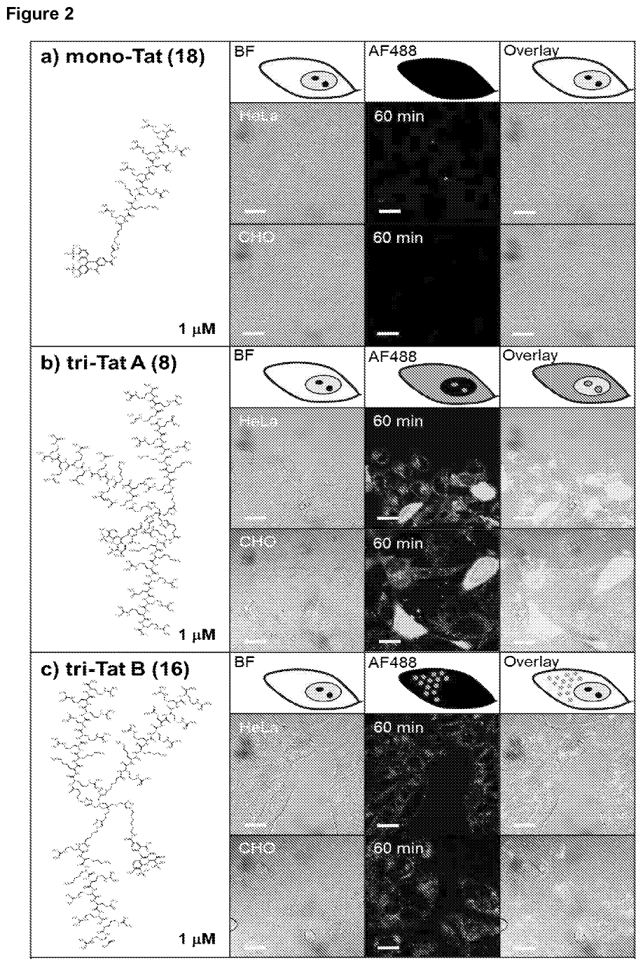

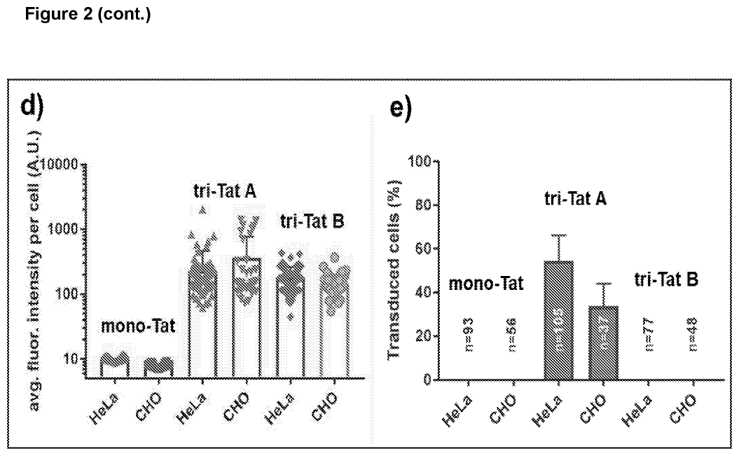

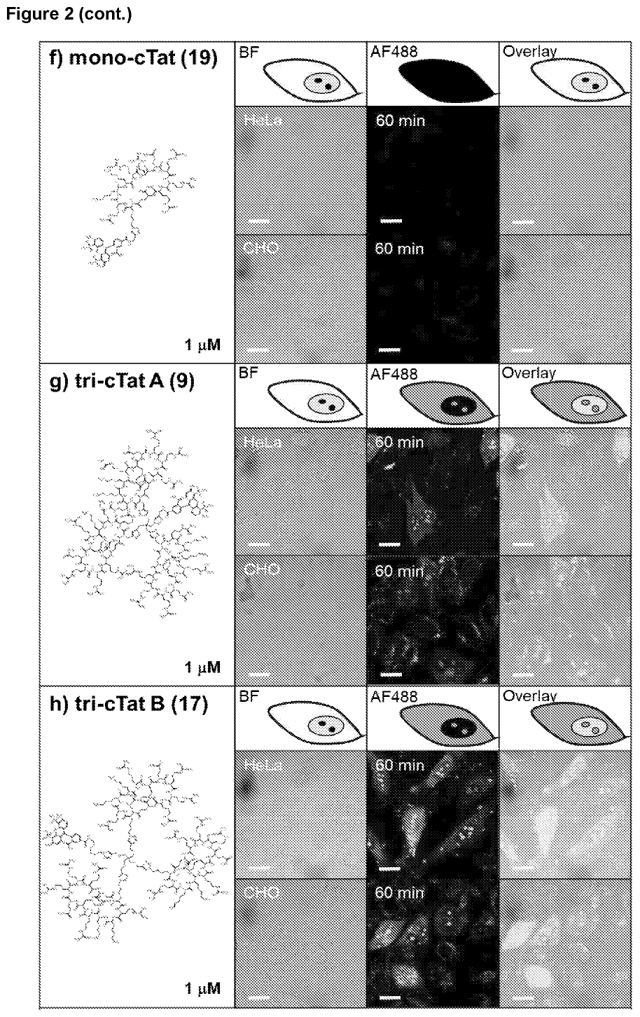

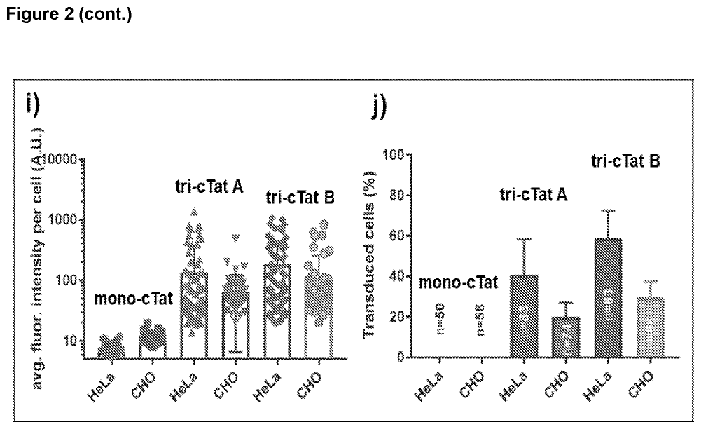

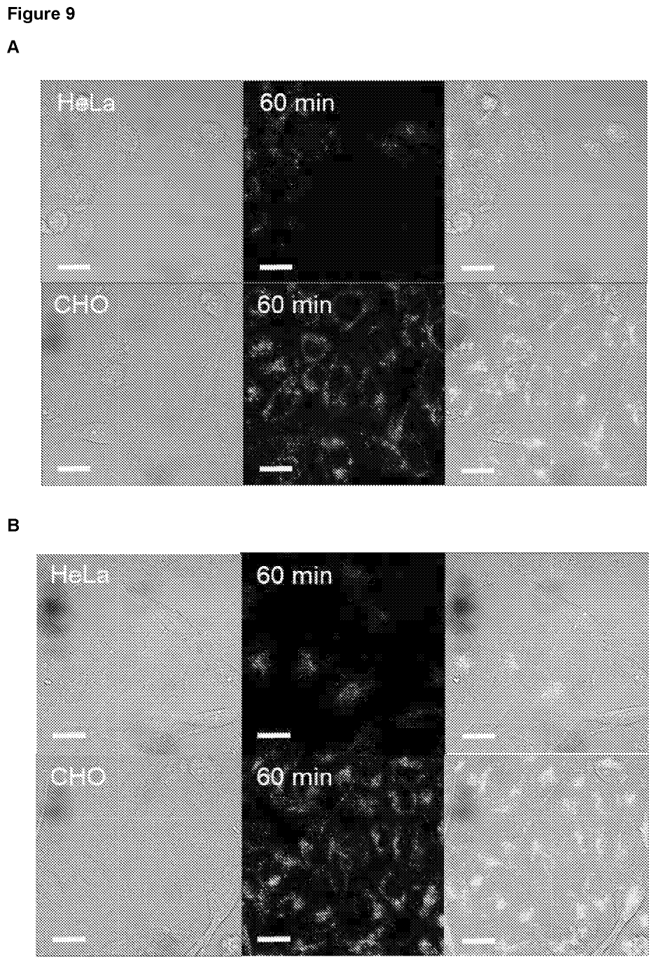

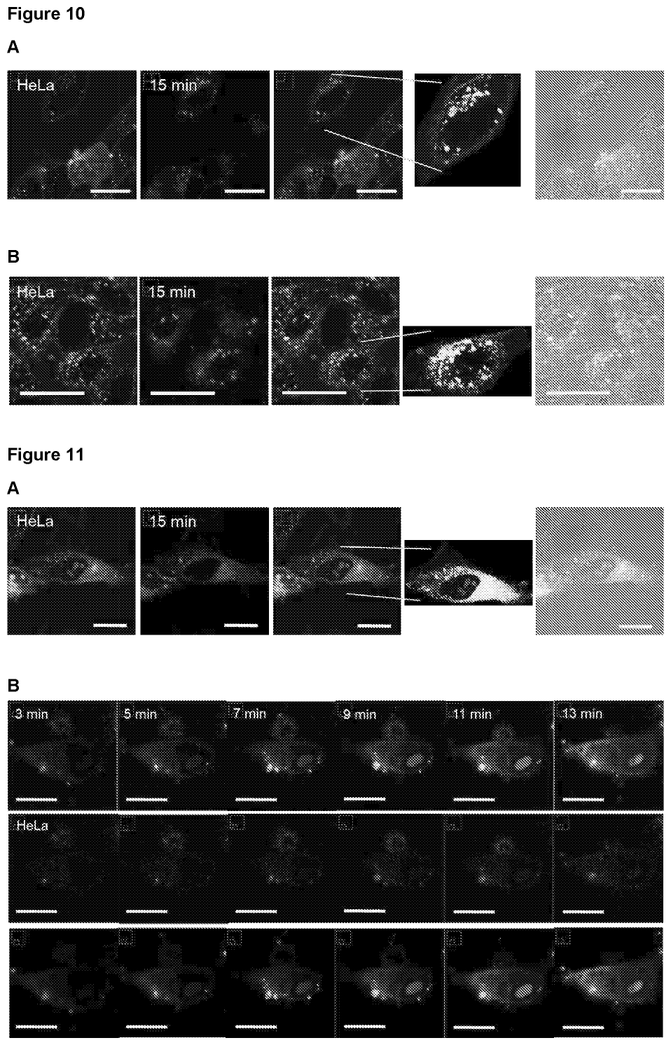

The cellular uptake and intracellular distribution of Tat-trimers and monomeric controls was investigated by performing live cell confocal microscopy of HeLa (human, cervical cancer) and CHO (Chinese hamster, epithelial) cells incubated with Tat conjugates (1 μM) in serum-free DMEM (60 min). Following washing, images were acquired using a Zeiss 780 confocal microscope fitted with an incubation chamber (FIG. 2). In accordance with observations that free Tat in the cytosol readily enters the nucleus and strongly binds to nucleoli,²⁹ homogenous staining of cytoplasm and nucleoli were considered as indicative of cells having accumulated free conjugate, not confined to endosomes, for quantification purposes. The results show that mono-Tat (1 μM) is not taken up by HeLa or CHO cells (FIG. 2a). In contrast tri-Tat A, shows homogenous cytosolic uptake and nucleolar staining at this concentration (FIG. 2b). This effect is not due to the increased total amount of Tat peptide, as experiments with 10 μM mono-Tat indicate no cytosolic uptake or nucleolar staining (FIG. 9a). The uptake and intracellular distribution of tri-Tat A is similar in live Hela and CHO cells (FIG. 2b,d,e). Interestingly, experiments with tri-Tat B showed no evidence of cytosolic uptake or nucleolar staining in either HeLa or CHO cells (FIG. 2c,e). Experiments in Hela cells transfected with the early endosomal marker rab5a tagged with RFP (rab5a-RFP) confirm that the intracellular punctuate fluorescence displayed by tri-Tat B is due to endosomal entrapment (FIG. 10).

Similar to mono-Tat, and despite reports that cyclic Tat is more effective in comparison to linear Tat, mono-cTat is not taken up into HeLa or CHO cells (FIG. 2h). It is probable that differences between monomeric linear and cyclic Tat are only observed above certain threshold concentrations and thus not observed at 1 μM or 10 μM (FIG. 9b). By contrast, tri-cTat A (1 μM) shows homogenous cytosolic and nucleolar staining (FIG. 2g), although the staining intensity is lower than that observed with linear tri-Tat A, and transduction efficacy is slightly reduced (FIG. 2i,j). Interestingly, tri-cTat B appears to be more effective at cellular transduction in comparison to tri-cTat A both by average fluorescence intensity per cell and percentage of transduced cells. As cell transduction and amount of trimer delivered into the cell differ for linear versus cyclic trimers, we propose that cyclic timers enter the cytosol by a different mechanism from linear trimers. These initial experiments identified linear tri-Tat A and cyclic tri-cTat B as the most promising lead compounds.

### Example 3. Time Course of Uptake in Hela Cells

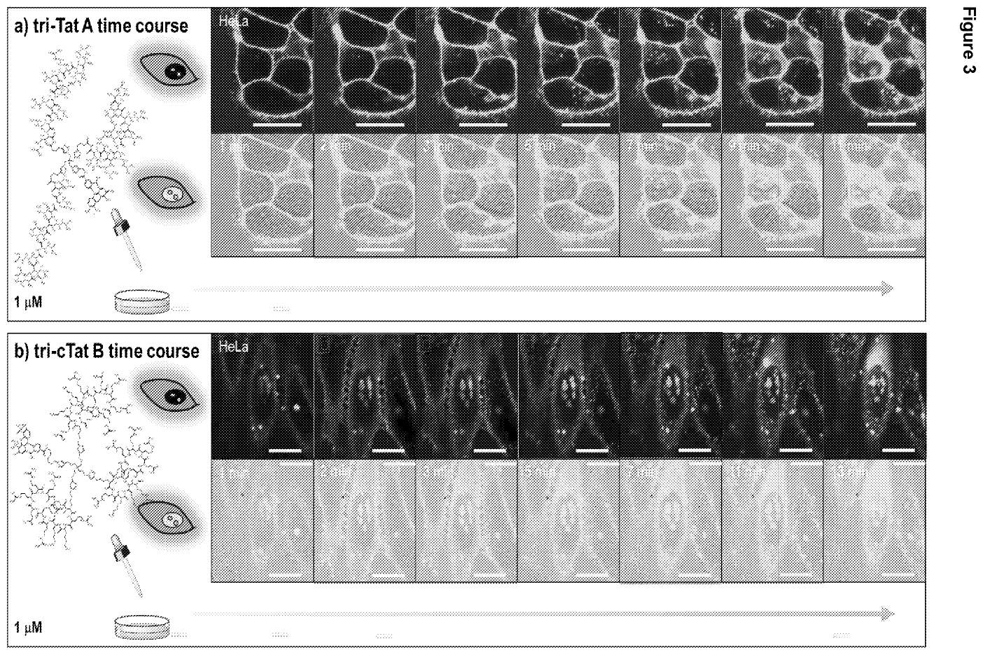

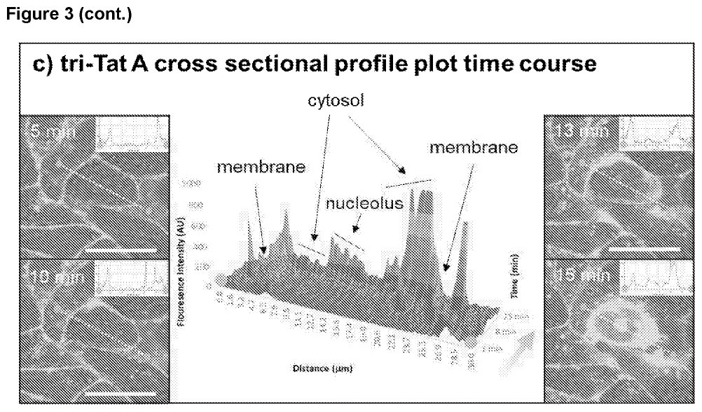

To better understand the mechanism by which tri-Tat A and tri-cTat B enter the cytosol and the nucleus, we investigated the uptake kinetics of the conjugates during the first 15 min after their addition to Hela cells using confocal live cell microscopy. Tri-Tat A (FIG. 3a) or tri-cTat B (FIG. 3b) (1 μM) were added to the cells and images acquired from 1 min to 15 min at 60 sec intervals. The two trimers show distinct uptake patters into Hela cells; to better understand the difference in uptake kinetics, we drew cross sectional profile plots through representative cells treated with tri-Tat A (FIG. 3c) and tri-cTat B (FIG. 3d). These plots reveal initial strong association of tri-Tat A with the membrane, indicated by two signals on either end of the plot, and no fluorescence in the cytosol; fluorescence signal then spreads from the membrane into the cytosol. In contrast, Tri-cTat B displays little association with plasma membrane and instead forms large vesicular bodies in the cytosol; fluorescence signal then spreads from these vesicles into the cytosol. To investigate whether findings in individual cells also apply to larger populations, we extended the plot profile analysis by designating regions of interest (ROI) corresponding to plasma membrane, cytosol (excluding endosomes), nucleus, and nucleoli along the cross section, using information from brightfield images.³⁰ Average fluorescence intensities for each ROI, at each time point, in six cells per treatment group were calculated and plotted in FIGS. 3e-3h. Tri-Tat A shows significant, approximately three fold stronger association with the plasma membrane (p<0.05 for t=1 to 15 min; FIG. 3a;). While the cytosolic signal of tri-cTat B gradually increases over time, cells treated with tri-Tat A, show little cytosolic accumulation (significantly lower than tri-cTat B from 4 to 7 min (p<0.05; SI), until signal increases rapidly after ˜12 min and exceeds tri-cTat B signal by a factor of 2 at 15 min (p<0.05, SI). Similar patterns are observed for accumulation in the nucleus and nucleoli. These observations are consistent with different mechanism of cell entry for the two trimmers. Tri-Tat A translocates directly across the membrane, rather than or in addition to entry into the cytosol via endosomal escape, while the route of cell entry for tri-c Tat B is the formation of vesicle-like bodies followed by vesicular escape.

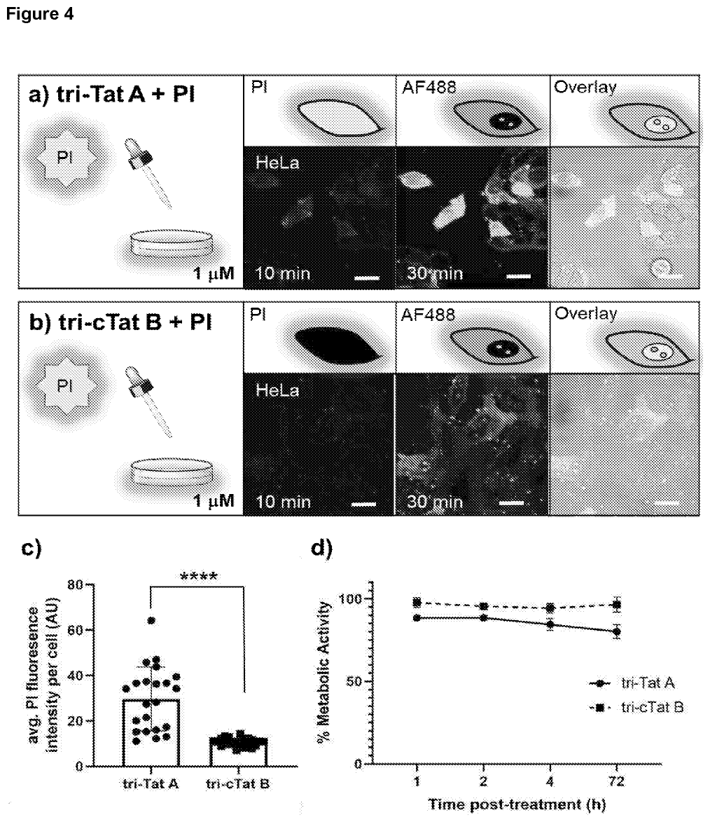

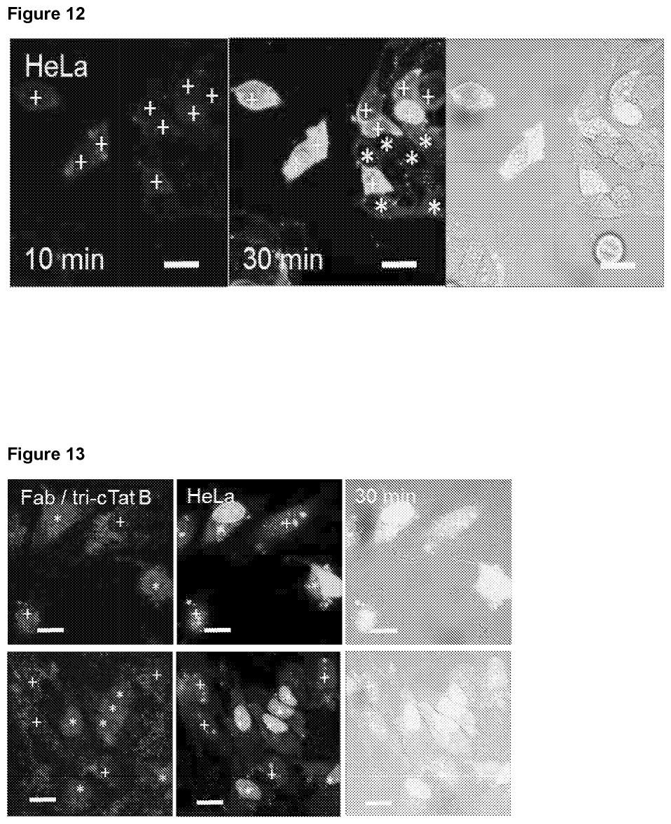

To assess membrane integrity, 40 μM propidium iodide (PI; a cell impermeable dye), was added to Hela cells 20 min after the addition of the peptide trimer. Cells with free tri-Tat A in the cytosol also contain PI (FIG. 4a), indicating that transient pores are formed, once a critical peptide to membranous lipid ratio is reached, allowing the diffusion of PI (FIG. 12). The amount of PI in cells, as measured by average fluorescence signal in the cytosol and nucleus, is significantly higher in cells treated with tri-Tat A than tri-cTat B (p<0.0001; FIG. 4b, c), indicating that the cyclic peptide trimer does not form pores in the plasma membrane. Co-localization of tri-cTat B and rab5a confirms that the vesicle-like structures are endosomes; rab5a associates with endosomes before release of cargo into the cytosol (FIG. 11).

Toxicity is a concern with potent intracellular delivery agents. We used an MTT assay to measure the metabolic activity of Hela cells treated with 1 μM tri-Tat A or tri-cTat B for 60 min (FIG. 4d). Tri-cTat B shows little acute toxicity (cells 94.3% viable, 4 h post-treatment), and no chronic toxicity (cells 96.5% viable, 3 days post-treatment). Tri-Tat A is slightly more toxic, which is consistent with the observation that the trimer forms membrane pores (cells 84.4% viable, 4 h post-treatment; 80.2% viable, 3 days post-treatment). The favourable uptake mechanism and low toxicity of tri-cTat B lead us to investigate this agent for its potential to transport biomacromolecular cargos into cells.

### Example 4. Co-Delivery of Antibodies and Antibody Fragments

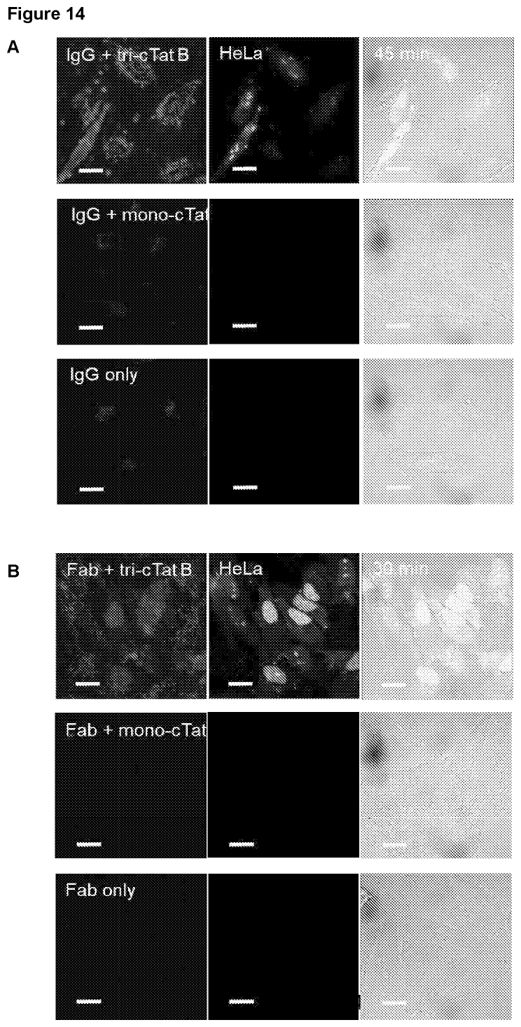

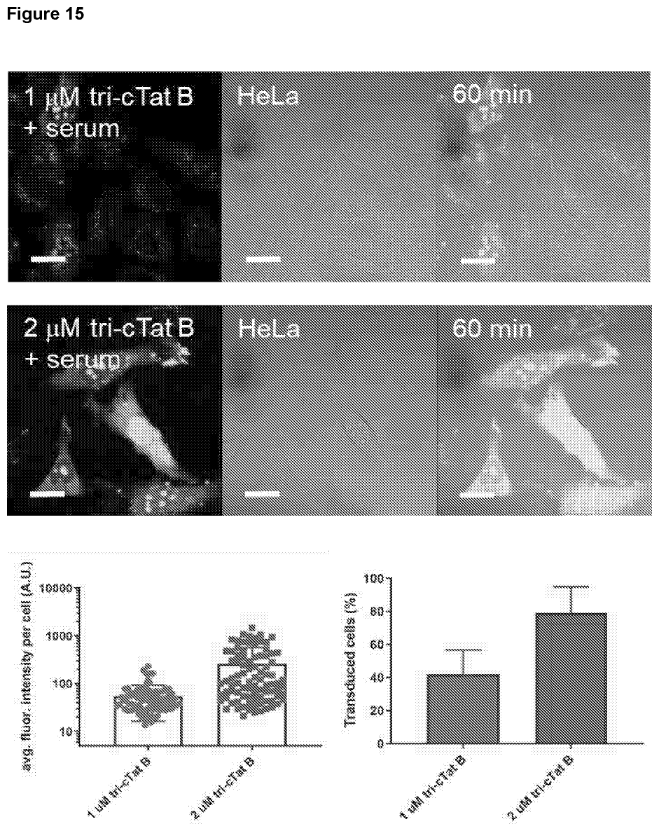

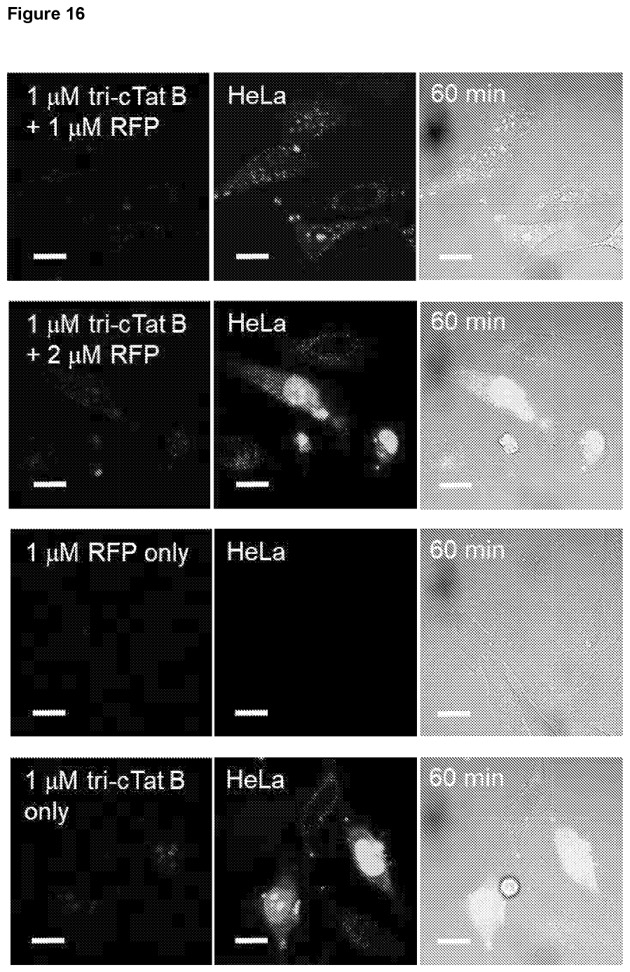

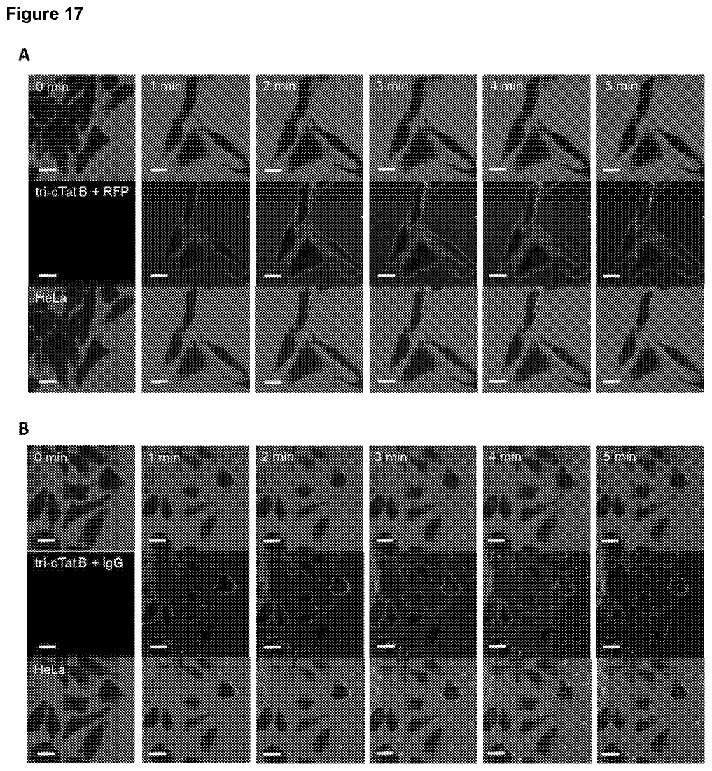

Access to the cytosol by mechanisms that are independent of transmembrane pore formation constitutes a preferred method of co-delivery due to reduced toxicity to the cell. Treatment of Hela cells with 500 nM AF647-labelled non-specific mouse Fab fragment and 1 μM tri-cTat B for 30 min resulted in successful delivery of Fab into cytosol and nucleus of 50% of cells in a typical experiment (FIG. 5a, d). Successful delivery of Fab is indicated by homogenous distribution of AF647 signal in the cytosol and nucleus and is only observed in cells which have been transduced with tri-cTat B (FIG. 13). Time course of Fab uptake by tri-cTat B (FIG. 5c) follows a similar pattern as tri-cTat B alone (FIG. 3b, d). Tri-cTat B and Fab form large vesicle-like bodies that appear to release their contents into the cytosol of the cell; unambiguous cytosolic staining by Fab is observed from 23 min. Interaction between peptide trimer and biomacromolecule, mediated by charge-charge interactions is essential for the accumulation of both in endosomes and subsequent release and delivery of cargo into cells. Delivery of relatively β-sheet rich, neutrally charged recombinant RFP was unsuccessful (FIG. 16) due to a lack of association between tri-cTat B and cargo (FIG. 17). On the other hand, interaction diminishes the efficacy of the peptide trimer due to charge masking, emphasising the need to maintain an appropriate peptide to cargo ratio for a given macromolecule. Experiments to explore the efficacy of tri-cTat B in the presence of serum supplemented medium underline this principle; while tri-cTat B continues to function in the presence of serum, the number of transduced cells diminishes (42% (serum) vs. 61% (serum free)), but can be restored by increasing tri-cTat B concentration to 2 μM (79% (serum); FIG. 15). Whole IgG co-delivery by tri-cTat B is possible, but less effective than the delivery of Fab, with approximately 20% of cells showing homogenous AF647 staining in cytosol and nucleus (FIG. 5b, d). This is likely due to the larger size of antibodies and also to the observation that antibodies have a greater tendency to aggregate in the presence of tri-cTat B compared to Fab fragments. There was no cellular uptake of Fab or IgG in the absence of tri-cTat B or in the presence of mono-Tat/mono-cTat (FIG. 14). Finally, we investigated whether it is possible to deliver functional antibodies and antibody fragments into the cytosol and nuclei of cells. β-actin was chosen as a proof-of-principle protein because of its abundance in the cell and formation of distinctive subcellular filaments. To clearly identify actin filaments, Hela cells were transfected with red fluorescent protein fused to actin (actin-RFP) before being incubated with β-actin antibody (mouse IgG_{2b}) or β-actin Fab fragment (mouse) conjugated to AF647 plus tri-cTat B for 30 min. After washing, cells were left for 60 min to allow IgG/Fab to bind to actin filaments. FIG. 6a shows a cell transfected with actin-RFP (shown in orange (O)), transduced with tri-cTat B and co-delivered β-actin antibody fragment-AF647 (shown in red (R)), while FIG. 6b shows the co-delivery of β-actin IgG-AF647 into a similarly treated cell. To enable objective quantification of co-localization between β-actin filaments and IgG/Fab fragment (white arrows), a co-localization threshold algorithm was used in selected regions of interest (ROI). Pearson's and Manders' coefficients were obtained under Costes' automatic threshold³² and the co-localized pixels are shown as a mask of yellow pixels of constant intensity (co-localization panels). The significance of the co-localization parameters (Pearson's and Manders' coefficients) was evaluated using the confined displacement algorithm (CDA). This analysis indicates that approximately 4 to 8% of RFP signal in ROIs co-localizes with IgG/Fab; while approximately one third of IgG/Fab signal co-localizes with β-actin RFP, resulting in statistically significant correlation in the areas indicated (white arrows, yellow mask). These results suggest that β-actin Fab fragments and IgG antibodies retain their ability to bind actin stress filaments in the cytoplasm of cells.

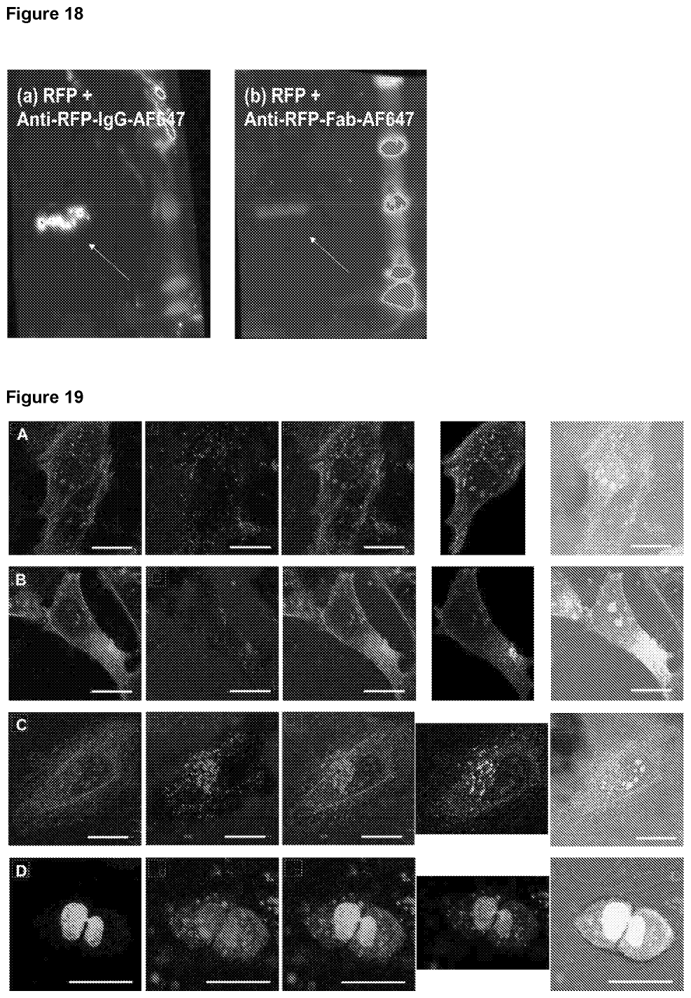

We went on to investigate whether functional antibodies can be delivered to the nucleus using tri-cTat B. Hela cells were transfected with a histone-RFP fusion protein and then incubated with tri-cTat B and an anti-RFP IgG (mouse, IgG₁; FIG. 6c). The antibody enters the nucleus and co-localizes with RFP (white arrows). Co-localization analysis on the ROI indicated that approximately half of the RFP signal co-localizes with IgG and approximately 45% of IgG signal co-localizes with RFP, resulting in a significant correlation. Delivery of anti-RFP Fab fragment into the cytoplasm and nucleus of cells was accomplished, but co-localization with histone-RFP could not be demonstrated, due to low affinity of the Fab fragment for RFP protein (FIG. 18). The remainder of intracellular antibody and Fab remains confined to vesicles that localize in the perinuclear region. Trafficking of IgG and Fab to the nucleoli was observed, an off-target effect mediated by charge-charge association with tri-cTat B (FIG. 19). The co-localization of antibodies and antibody fragments with actin filaments in the cytosol and RFP in the nucleus demonstrates that macromolecules delivered using tri-cTat B retain their ability.

The proximity ligation assay (PLA; Duolink®) which allows optical detection of protein-protein interactions (PPI) was used to show that co-localization of CPP internalised antibodies and targeted RFP-tagged proteins results from specific interaction of the two. Samples are first incubated with antibodies against the two target proteins and then incubated with secondary antibodies with attached oligonucleotides that form a circular DNA structure only when the two primary antibodies are within 40 nm of each other. The circular DNA is amplified through addition of a complementary sequence that incorporates a fluorophore and thus reveals the presence of the PPI and its subcellular localization PLA detects protein-protein interactions in their native form compared to other techniques such as co-immunoprecipitation, which requires disruption of the cell structure, or FRET, which requires extensive data processing. Here, the interaction between an anti-H2B-AF488, co-delivered to the cytosol and nuclei of Hela cells using tri-cTat B lacking a fluorophore label (cTat3-alkyne, 14), and an H2A.Z antibody, introduced in the cells after permeabilization, is observed. Proximity detection by secondary antibody-mediated oligonucleotide ligation followed by rolling-circle amplification (RCA) reveals red spots in the nuclei of cells that also show nuclear accumulation of internalised H2B-AF488 IgG (FIG. 20a). The PLA signal in cells incubated with H2B-AF488 IgG plus tri-cTat B and then with H2A.Z IgG was significantly higher than in cells incubated with either H2B-AF488 or H2A.Z IgG alone (FIG. 20b; p<0.01). This provides evidence that anti-H2B IgG delivered into the nucleus by tri-cTat B is functional and binds the H2B/H2A.Z dimer that constitutes part of the nucleosome octamer. These results validate the findings of the co-localisation experiments and show that antibodies and antibody fragments delivered into the cytosol and nucleus using tri-cTat B retain their ability to bind target proteins.

### Discussion

The arrangement of Tat peptides into multimeric clusters on a defined chemical scaffold provides delivery agents that are significantly more effective than previously reported CPPs. The significant difference in transduction efficacy between the linear trimers tri-Tat A and tri-Tat B is surprising, but broadly consistent with reports suggesting that higher charge density leads to more effective uptake into cells. The interaction between trimer geometry and cell surface clusters might account for differences in uptake efficacy between different cell lines and raises the possibility of enhancing uptake into specific cell types by altering CPP cluster geometry. The effect of multimerization on binding efficacy to cell surface proteins has recently been demonstrated.

Despite reports that cyclisation of CPPs can increase efficacy by up to two orders of magnitude, cyclic trimers tri-cTat A and tri-cTat B did not show significantly improved uptake into the cytoplasm and nuclei of cells compared to linear Tat trimers. Instead, cyclisation of Tat peptide on the trimer scaffold leads to a shift in mechanism from direct interaction with the plasma membrane to vesicular escape. A model, proposed by Pei and co-workers, argues that there is no mechanistic difference between linear and cyclic peptides of the same sequence and postulates an exclusively quantitative difference. The results of the current study however suggest that there is a qualitative difference in mechanism of uptake; trimerization of linear peptides and their cyclic equivalents likely add a further element of complexity to the behaviour of CPPs in vitro. Two features of tri-cTat B uptake are broadly consistent with the proposals of Pei et al: (1) the formation of relatively large vesicle-like bodies containing trimer and (2) the persistence of those vesicles while peptide spreads freely into the cytosol and nucleus.

The data presented in this report indicate that, rather than access to the cytosol by a single pathway, CPPs are capable of binding a variety of cell surface components and are apt to switching between uptake mechanisms to find the most energetically favourable path into the cell. Our results show that it is possible to alter the mechanism of uptake by changing the geometry of conformation of Tat trimers, while leaving amino acid sequence and net charge unaltered.

It has been shown previously, that endosomal escape is an efficient and non-toxic mechanism for intracellular cargo delivery. Successful co-delivery of antibodies and antibody fragments using cyclic tri-cTat B, is consistent with this. Our results further suggest that an appropriate peptide to cargo ratio is crucial for successful co-delivery. Charge-charge association between peptide and cargo is necessary for the formation of endosomes containing both agents followed by release, but also leads to charge masking and reduced efficacy of the peptide. These opposing effects need to be balanced for efficient delivery of macromolecules into cells. The relative structural homogeneity of antibodies and their fragments warrants the optimization of delivery protocols that are broadly applicable to these classes of cargo. Co-delivery of structurally diverse proteins is more challenging, as the results for RFP demonstrate. However, the absence of charge-charge interaction is likely to be a considerable advantage for covalently linked peptide-cargo conjugates, where high efficacy of delivery can be maintained, as previously demonstrated.

The co-localization of protein targets and targeting agents in live cells provides insight into the efficacy of immunoglobulins in intracellular environments. While the fraction of cargo bound to target is relatively high at 30% to 50%, the fraction of target occupied by cargo is dependent on the nature and abundance of the target. β-actin is a high abundance protein (>10 μM), with only a small fraction expressed as RFP fusion protein. Intracellular cargo concentrations are at least 1 magnitude lower than target concentration; consequently, target occupancy is low (<10%). In contrast, histone-RFP expression is low, leading to relatively low abundance of RFP in the nucleus; as a result, higher target occupancy is observed (˜45%). These observations, combined with the sub nanomolar affinity of immunoglobulins for their targets, suggest that this approach might be most suited to low abundance targets.

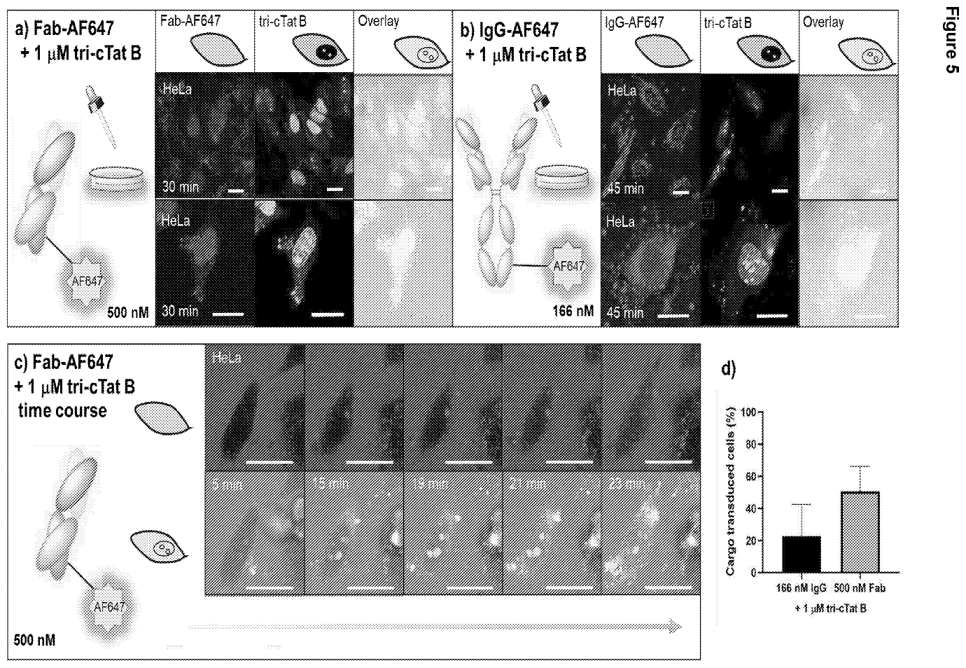

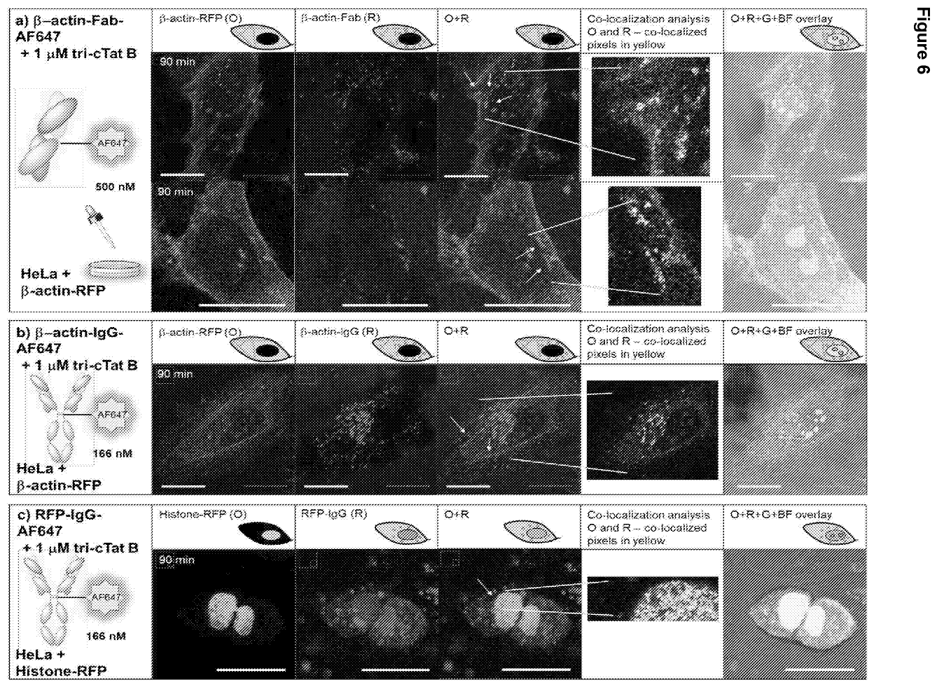

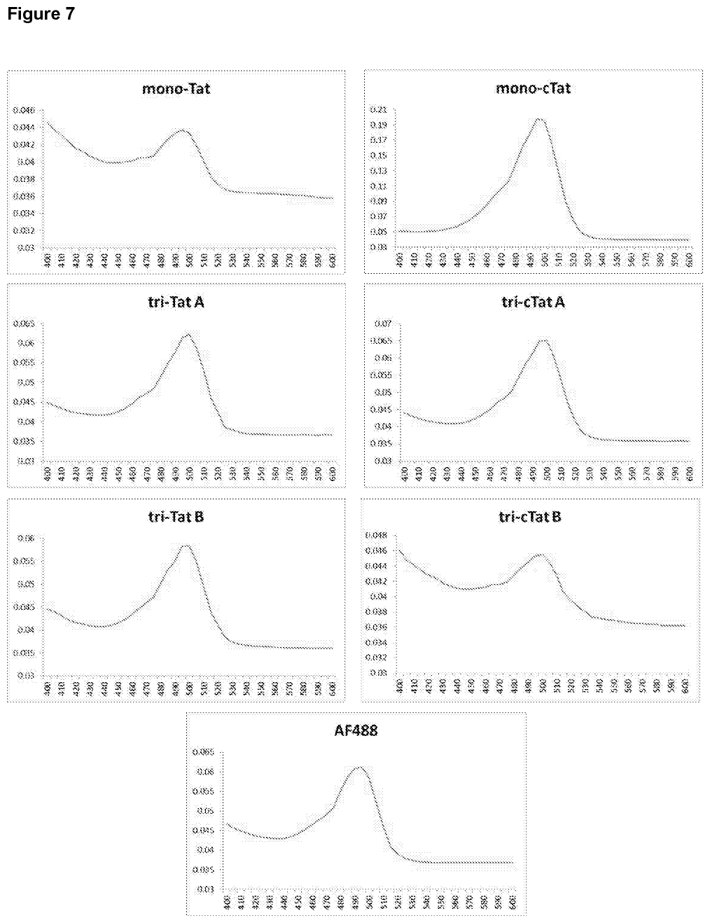

Proximity ligation assays detected binding of H2B-AF488 IgG, internalised in the presence of tri-cTat B, to the H2B/H2A.Z dimer that constitutes the histone octamer in the nucleus, demonstrating successful nuclear targeting and validating co-localization data. The mechanism by which antibodies enter the nucleus is not known. However, the ability of antibodies to enter the nucleus has been reported previously, particularly anti-DNA autoantibodies. Several investigators have demonstrated nuclear uptake of IgG molecules modified by the addition of oligonucleotides or nuclear localising signal (NLS). Our data points to continuing interaction between tri-cTat B and IgG cargo in the cytosol of cells. It is possible that this association allows nuclear delivery of cargo using the NLS of the Tat peptide. Large cargos such as pathogens and pre-ribosomes (some up to 40 nm in diameter) are able to cross from the cytoplasm into the nucleus, suggesting that, based on size, whole IgGs which have a length of approximately 15 nm, could also do so. As well as intranuclear signal a few spots of fluorescence were located in the cytoplasm in the proximity ligation assay (FIG. 7a). Histones, like other proteins, are synthesised in the cytoplasm, and are then transported into the nucleus as dimers (including H2A.Z-H2B). Therefore, a small amount of H2B and H2A.Z dimer does exist in the cytoplasm and was detectable in the proximity ligation assay following tri-cTat B-mediated internalisation of H2B-AF488 IgG.

The successful targeting of actin filaments using β-actin antibodies and Fab fragments, as well as intranuclear RFP using an IgG, raises the possibility of using immunoagents against therapeutic targets inside the cytoplasm and nucleus to affect previously undruggable disease relevant pathways. Tri-cTat B is an efficient non-viral delivery agent allowing the rapid, non-toxic transport of biomacromolecular cargo into cells. This approach has some important advantages over viral delivery methods, which are limited to nucleic acid delivery and can trigger host immune responses, as well as mechanical delivery methods, which frequently lead to loss of cytoplasmic content, can excessively damage organic molecules, denature proteins, and are less amenable to in vivo translation. In summary tri-cTat B can be used as an effective delivery agent to address the challenge of targeting intracellular therapeutic targets. Furthermore, the structure-activity insights reported here will aid the development of novel CPP multimers with tailored mechanistic properties.

### Tat Trimers C, D, E and F

### Example 5. Synthesis of Tat Trimers C, D, E and F

Four further Tat trimers were designed using four different PEG-alkyne scaffolds furnished with either linear or cyclic Tat (cTat) peptide. These were chosen to determine to determine whether the efficacy of cyclic trimers decreases with increasing scaffold flexibility and the maximum permissible PEG size with sub-micromolar efficacy. Tat-trimers C, D, E and F were synthesized by copper catalysed 2+3 cyclo-addition between azide and alkyne functionalized components. The synthesis of trimers tri-Tat C, D, E and F and tri-cTat C, D and E is based on the following PEG-alkyne functionalised scaffold: C—[CH₂—(O—CH₂—CH₂—O—)ₘ—CH2—CH2—O—C≡CH]₄, wherein m=1, 2, 3 and 4 for trimers C, D, E and F, respectively.

Linear Tat (49-57) peptide (RKKRRQRRR), modified with an azido-pentanoyl (azide) (11—FIG. 1f) at the N-terminus was used to synthesize tri-Tat C, D, E and F (20-23). Cyclic Tat was custom designed to match the linear sequence; azide functionality was added by including an azide modified lysine (K(N₃)) residue at the N-terminus. Peptides were cyclized by addition of glutamic acid to the C-terminus for head to tail cyclisation to the N-terminus to generate cyclic K(N₃)RKKRRQRRRG (12). This peptide was used to synthesize cyclic trimers tri-cTat C, D and E (24-26).

Each of the Tat-trimers were labelled with AlexaFluor488 (AF488) for further study.

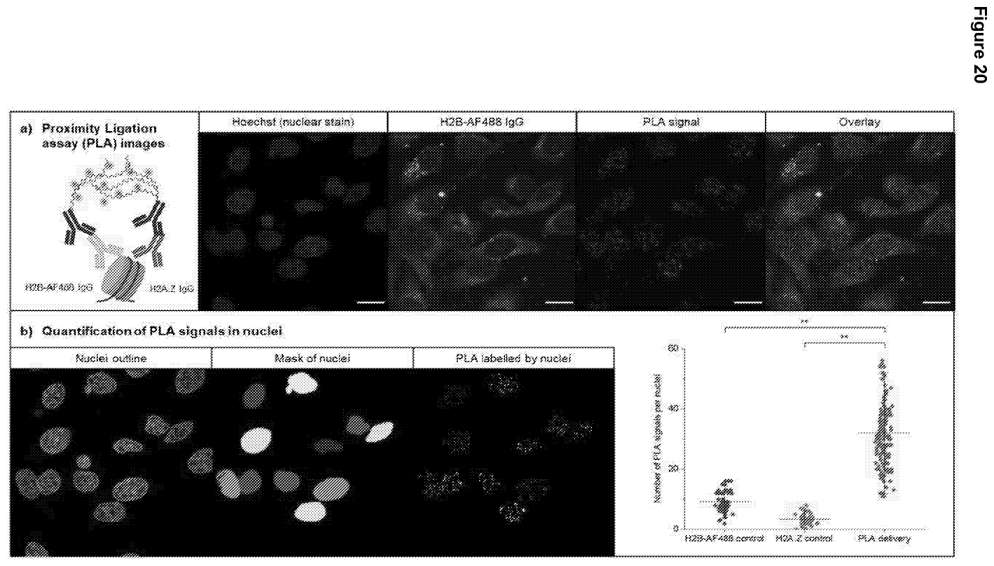

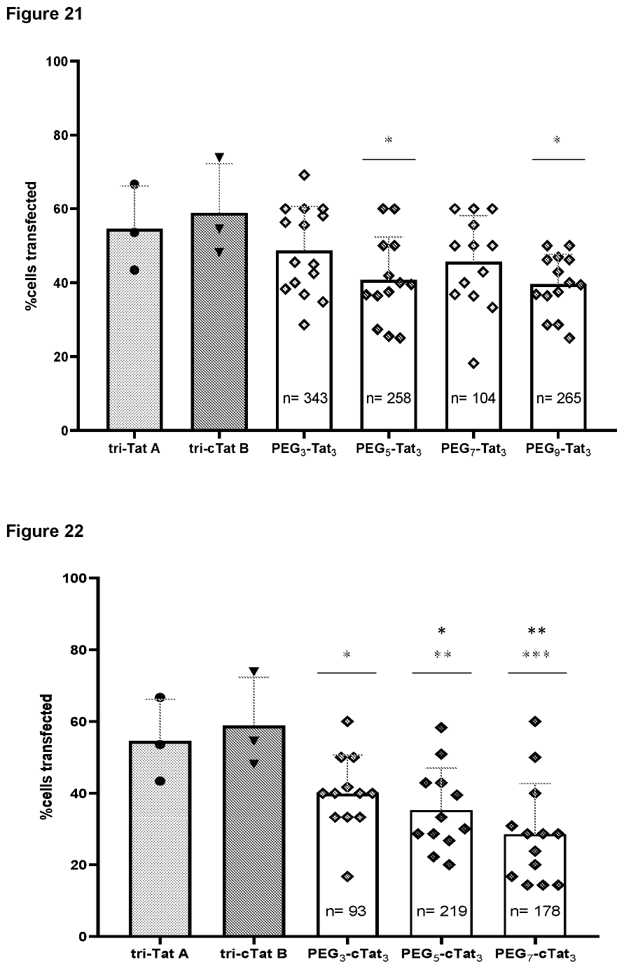

The Tat trimers for this study are summarised in the Table below, wherein “m” refers to m of the scaffold, C—[CH₂—(O—CH₂—CH₂—O—)ₘ—CH2—CH2—O—C≡CH]₄:

| | | | Com- | | |
|---|---|---|---|---|---|
| | | No. PEG | pound | Linear/ | |
| | m | moieties | No. | cyclic | Ref in FIGS. |
| tri-Tat C | 1 | 3 | 20 | Linear | PEG₃-Tat₃ (FIG. 21) |
| tri-Tat D | 2 | 5 | 21 | Linear | PEG₅-Tat₃ (FIG. 21) |
| tri-Tat E | 3 | 7 | 22 | Linear | PEG₇-Tat₃ (FIG. 21) |
| tri-Tat F | 4 | 9 | 23 | Linear | PEG₉-Tat₃ (FIG. 21) |
| Tri-cTat C | 1 | 3 | 24 | Cyclic | PEG₃-cTat₃ (FIG. 22) |
| tri-cTat D | 2 | 5 | 25 | Cyclic | PEG₅-cTat₃ (FIG. 22) |
| tri-cTat E | 3 | 7 | 26 | Cyclic | PEG₇-cTat₃ (FIG. 22) |

### Example 6. Live Cell Uptake of tri-(c)Tat C, D, E and F

The cellular uptake and intracellular distribution of Tat-trimers was investigated by performing live cell confocal microscopy of HeLa (human, cervical cancer) cells incubated with Tat conjugates (1 μM) in serum-free DMEM (60 min). Following washing, images were acquired using a Zeiss 780 confocal microscope fitted with an incubation chamber. In accordance with observations that free Tat in the cytosol readily enters the nucleus and strongly binds to nucleoli,²⁹ homogenous staining of cytoplasm and nucleoli were considered as indicative of cells having accumulated free conjugate, not confined to endosomes, for quantification purposes. The results were compared to tri-Tat A (8) and tri-cTat B (17).

The results show that each of conjugates 20-26 are able to enter the cells at 1 μM concentration and distribute homogenously in the cytoplasm and nucleus (FIGS. 21 and 22). There is not a significant difference between tri-Tat A (8) and any of the linear conjugates (tri-Tat C, D, E and F; 20-23).

### Methods

### Synthesis of Tat-Conjugates

Peptide and fluorophores were conjugated to tetrakis scaffolds via copper-catalysed azide alkyne cycloaddition reactions (CuAAC), using azide/alkyne starting materials and 1 eq. CuSO₄, 5 eq. tris((1-hydroxy-propyl-1H-1,2,3-triazol-4-yl)methyl)amine (THPTA), 2 eq. sodium L-ascorbate, 3 eq. aminoguanidine hydrochloride per cycloaddition, followed by HPLC purification and analysis by mass spectrometry.

Azide functionalized scaffold A (1,3-diazido-2,2-bis(azidomethyl)propane—2) was used to synthesize Tri-Tat A and tri-cTat A; alkyne functionalized scaffold B (tetakis (2-propynyloxymethyl)methane—10) was used to synthesize tri-Tat B and tri-cTat B; alkyne functionalized scaffold C—[CH₂—(O-CH₂—CH₂—O—)₁—CH2—CH2—O—C≡CH]₄ was used to synthesize tri-Tat C and tri-cTat C; alkyne functionalized scaffold C—[CH₂—(O—CH₂—CH₂—O—)₂—CH2—CH2—O—C≡CH]₄ was used to synthesize tri-Tat D and tri-cTat D; alkyne functionalized scaffold C—[CH₂—(O—CH₂—CH₂—O—)₃—CH2—CH2—O—C≡CH]₄ was used to synthesize tri-Tat E and tri-cTat E; and alkyne functionalized scaffold C—[CH₂—(O—CH₂—CH₂—O—)₄13 CH2—CH2—O—C≡CH]₄ was used to synthesize tri-Tat F. Linear Tat (49-57) peptide (RKKRRQRRR), modified with a hexynoyl (alkyne) (3) or an azido-pentanoyl (azide) (11) functional group at the N-terminus was obtained from International Peptides Inc., Louisville, KY, USA. Cyclic Tat was custom designed to match the linear peptide and synthesized by Cambridge Peptides, Birmingham, UK. Azide/alkyne functionality was added by including either an azide modified lysine (K(N₃) residue or propargyl glycine (Pra) at the N-terminus. Peptides were cyclized by addition of glutamic acid to the C-terminus for head to tail cyclisation to the N-terminus to generate cyclic Pra-RKKRRQRRRG (4) and K(N₃) RKKRRQRRRG (12).

The synthesis of tri-Tat A (8), tri-Tat B (9), tri-cTat A (16) and tri-cTat B (17), as well as mono-Tat (18) and mono-cTat (19) is summarized below (THPTA-Tris((1-hydroxy-propyl-1H-1,2,3-triazol-4-yl)methyl)amine; NaAsc-Sodium L-ascorbate; AminoGuan-Aminoguanidine HCl):

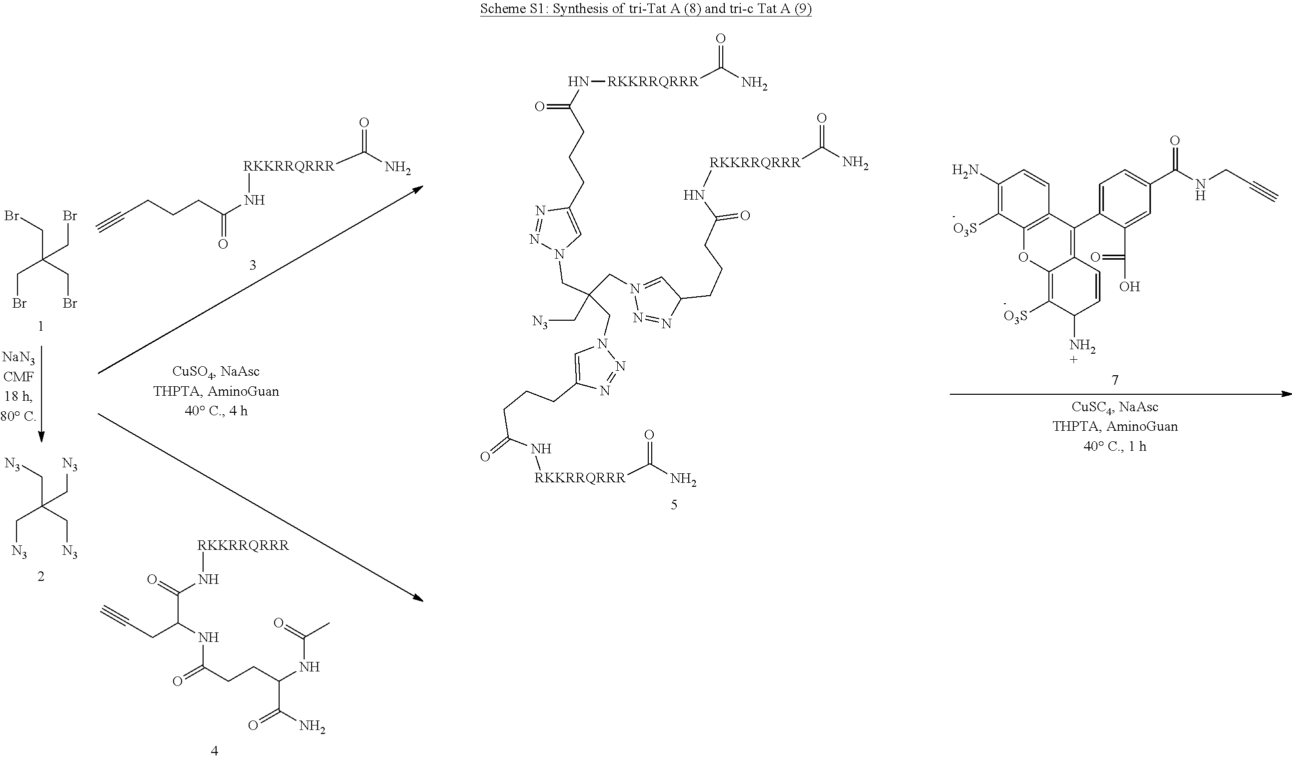

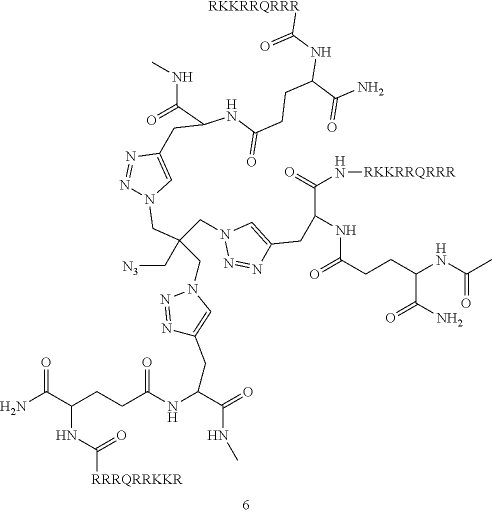

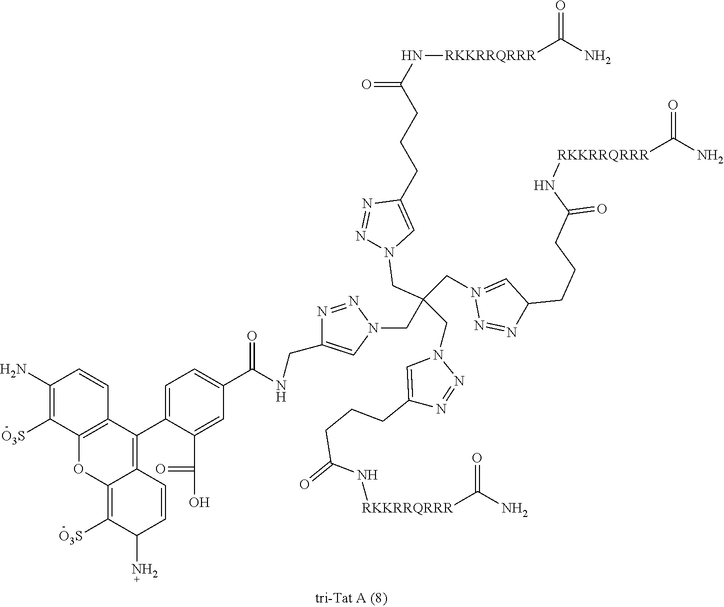

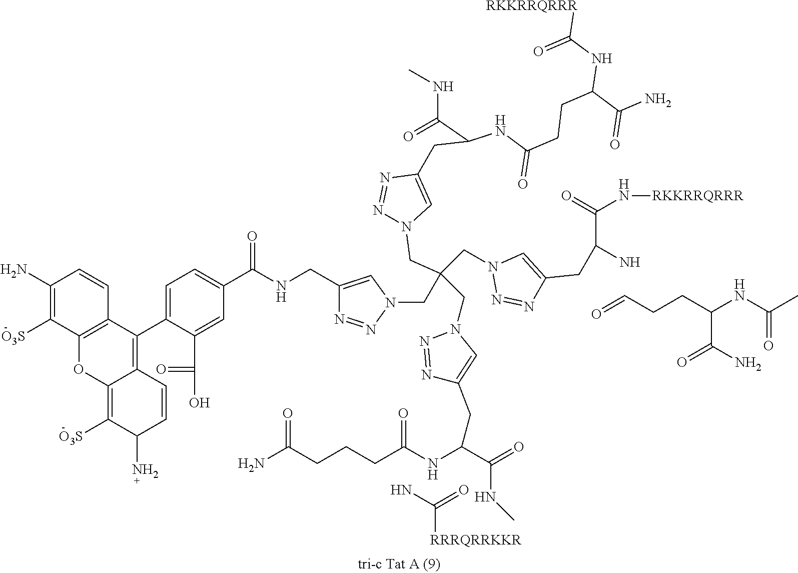

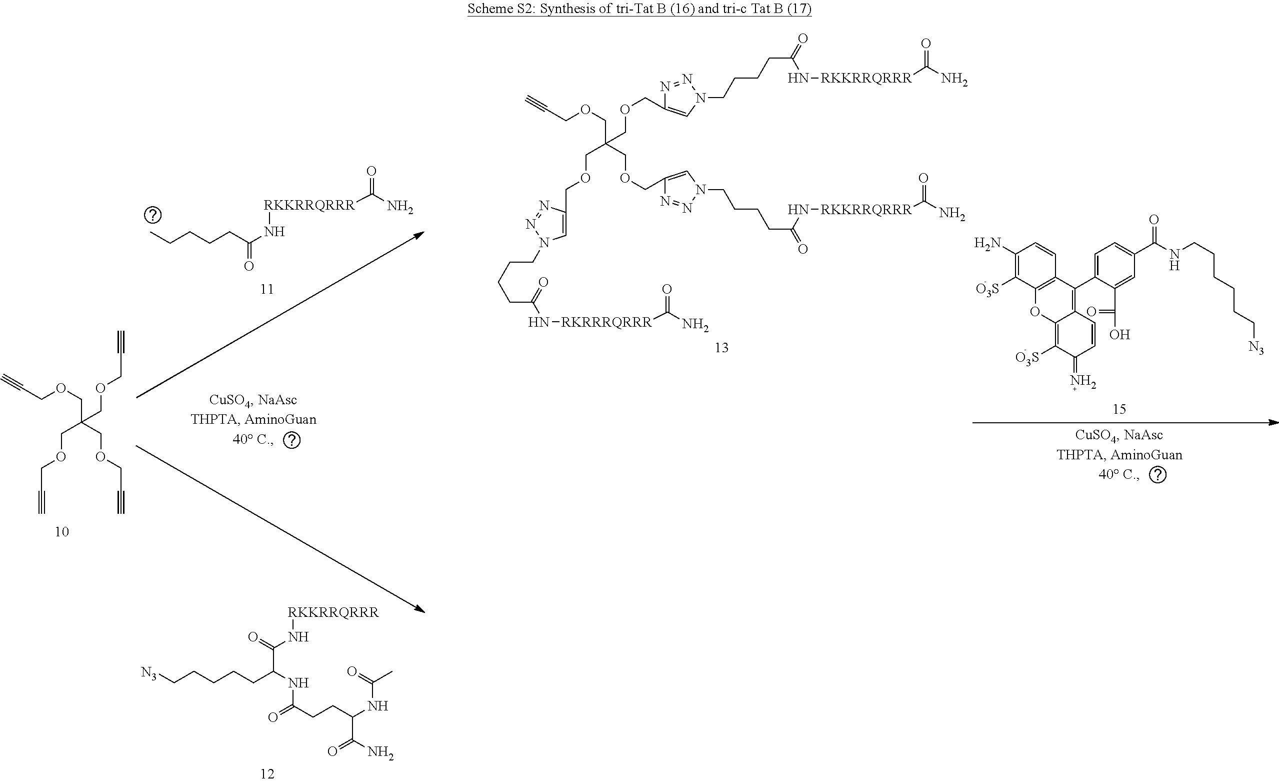

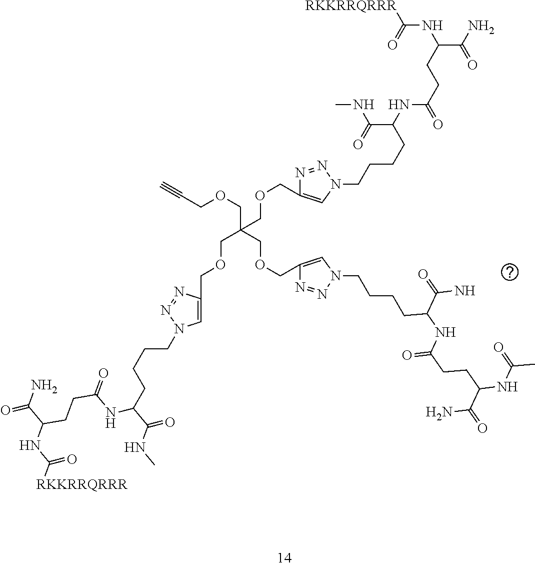

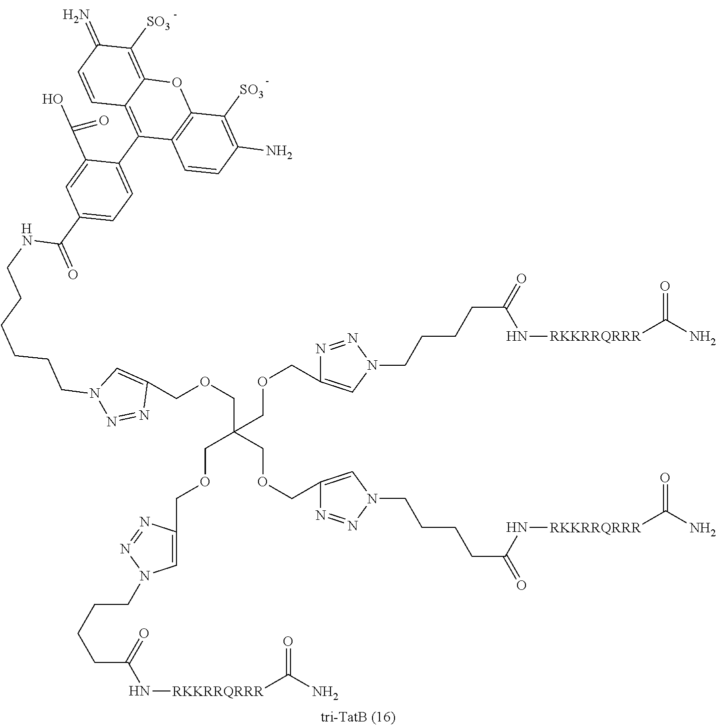

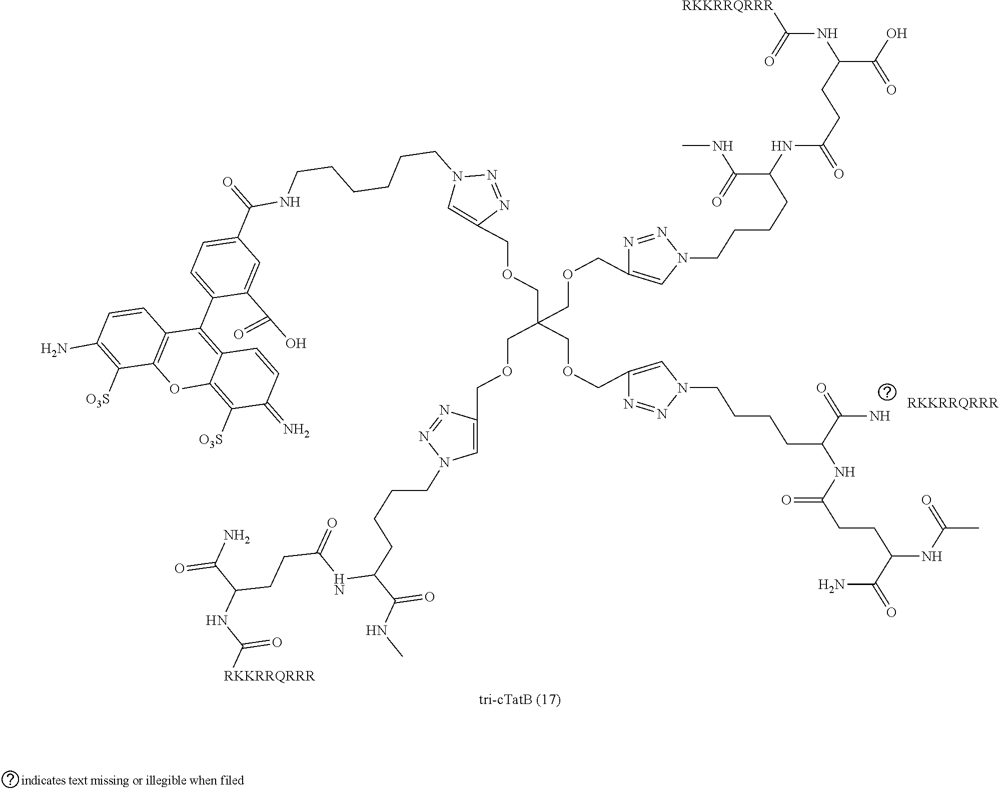

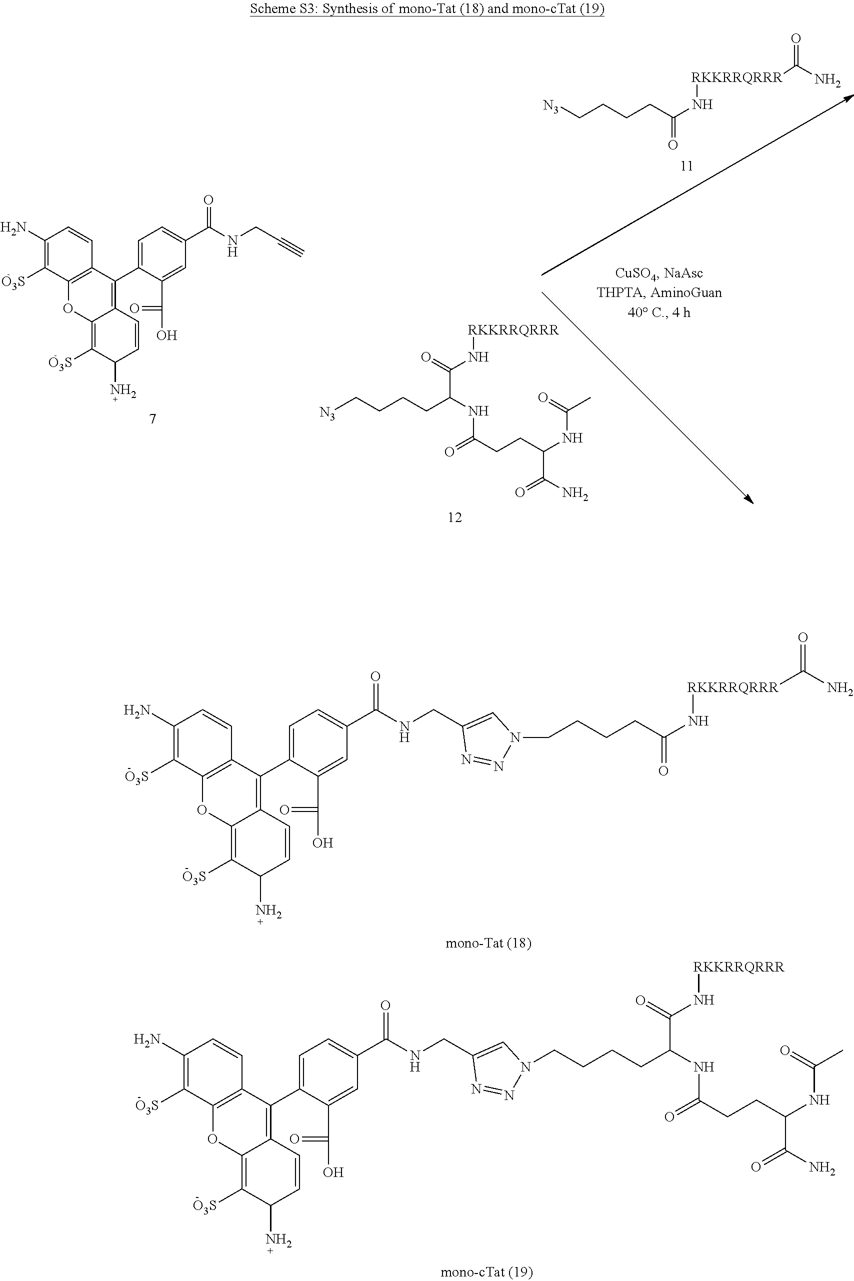

### General Procedure for Copper Catalyzed 2+3 Cycloaddition Reactions (Compounds 5, 6, 8, 9, 13, 14, 16-19)

Peptide and fluorophores were conjugated to tetrakis scaffolds using copper-catalyzed azide alkyne cycloaddition reactions (CuAAC). Optimized reaction conditions include shaking at 1,000 rpm and heating at 40° C. for 60 to 240 min using a thermoshaker. Molar ratios of reagents were normalized to the number of CuAAC per molecule and are quoted for each reaction specifically, in general 1 eq. CuSO₄, 5 eq. Tris((1-hydroxy-propyl-1H-1,2,3-triazol-4-yl)methyl)amine (THPTA), 2 eq. Sodium L-ascorbate, 3 eq. Aminoguanidine hydrochloride were used per CuAAC. CuSO₄, THPTA, Sodium L-ascorbate and Aminoguanidine HCl were dissolved in water immediately prior to use, AF488-azide and AF488-alkyne were dissolved in DMSO, peptides dissolved in water and used from frozen stocks. Reactions were carried out in Ca/Mg-free Dulbecco's phosphate-buffered saline (PBS) with 30% DMSO. Reactions were quenched using EDTA (final concentration 10 mM) and HPLC purified using a Waters XBridge C18 (3.5 μm, 4.6×150 mm) analytical column or an Agilent Polaris C18-A (3.0 μm, 4.6×150 mm) analytical column. HPLC purification and quality control samples were run using a 40 min method, flow rate=1 ml/min, gradient rising from 1% CH₃CN (0.1% TFA)/H₂O (0.1% TFA) to 40% CH₃CN (0.1% TFA)/H₂O (0.1% TFA) over the course of 40 min. Fractions containing compound of interest were neutralized using PBS and solvent exchanged using Amicon Ultra 0.5 ml centrifugal filters (Ultracel—3 k) (Merck Millipore) by washing with PBS once followed by three washes with H₂O.

### Synthesis Compound 2

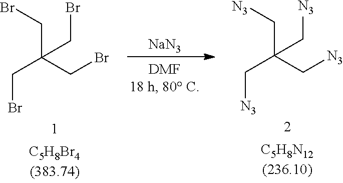 [SMILES: BrCC(CBr)(CBr)CBr ; [N-]=[N+]=NCC(CN=[N+]=[N-])(CN=[N+]=[N-])CN=[N+]=[N-] ; "NaN3"]

### 1,3-diazido-2,2-bis(azidomethyl)propane (2)

1,3-dibromo-2,2-bis(bromomethyl)propane (1) (200 mg, 0.52 mmol) and NaN₃ (170 mg, 2.60 mmol, 5 eq) were briefly dried under high vacuum and dissolved in DMF (5 ml). The reaction was heated under a constant flow of Argon at 80° C. for 18 h. The reaction mixture was separated between CHCl₃ and H₂O, the organic layer washed with H₂O (×3) and brine (×3) and dried over anhydrous Na₂SO₄. Solvent was removed in vacuo and the product purified by preparative TLC in 10% EtOAc/hexane (rf˜0.5). (NB: An azide stain was used to interpret analytical TLC plates, consisting of 1:1 propargylalcohol/EtOH and Cu(I)I, followed by treatment with a heat gun; azide containing compounds are stained white in comparison to brown stain on the rest of the plate.) The product was obtained as a white solid (100.3 mg, 0.425 mmol, 81.7% yield). ¹H-NMR (300 MHz, CDCl₃): 3.29 (s, 8H, 4×CH₂); ¹³C-NMR (75 MHz, CDCl₃): 44.1, 51.7; LCMS: 236.2 [ES+].

### Synthesis Compound 5

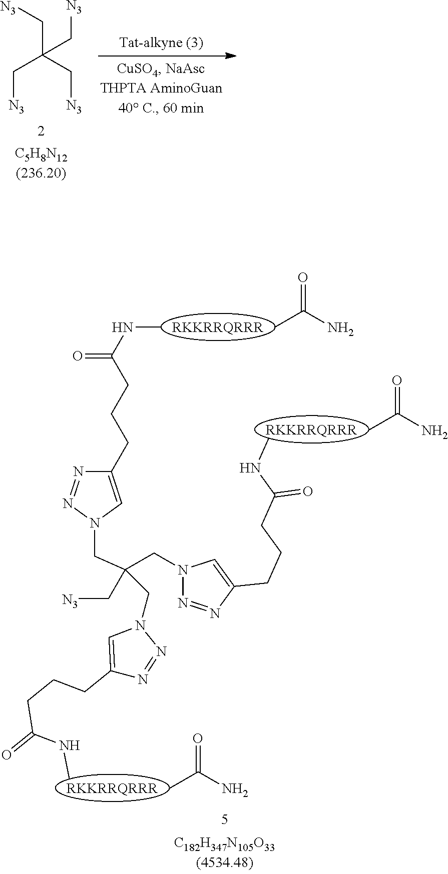

### Tat₃-N₃ (5)

1,3-diazido-2,2-bis(azidomethyl)propane (2) (38.5 μg, 0.16 μmol, 1 eq.) and 5-hexanoyl-Tat-NH₂ (—Tat-alkyne (49-57)) (Peptide International Inc., USA) (1000 μg, 0.70 μmol, 4.2 eq.) as well as CuAAC regents were dissolved in 30% DMSO/PBS and heated at 40° C. for 60 min. The product was purified by HPLC (294 μg, 0.06 μmol, 41% yield).

HPLC retention time rₜ=17:40 min; MS QTOF [ES+]: 4534.85 (calculated: 4534.48 for C₁₈₂H₃₄₇N₁₀₅O₃₃).

### Synthesis Compound 6

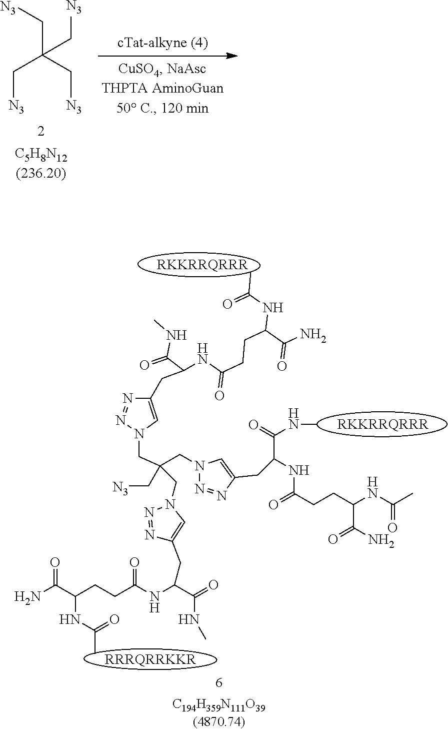

### cTat₃-N₃ (6).

1,3-diazido-2,2-bis(azidomethyl)propane (2) (35.8 μg, 0.15 μmol, 1 eq.) and propargyl-glycine-cyclicTat-NH₂ (4-cTat-alkyne) (Cambridge Peptides, UK) (1100 μg, 0.71 μmol, 4.7 eq.), as well as CuAAC regents were dissolved in 30% DMSO/PBS and heated at 50° C. for 120 min. The product was purified by HPLC (230 μg, 0.05 μmol, 31% yield).

HPLC retention time rₜ=19:16 min; MS QTOF [ES+]: 4871.88 (calculated: 4870.74 for C₁₉₄H₃₅₉N₁₁₁O₃₉).

### Synthesis Compound 8

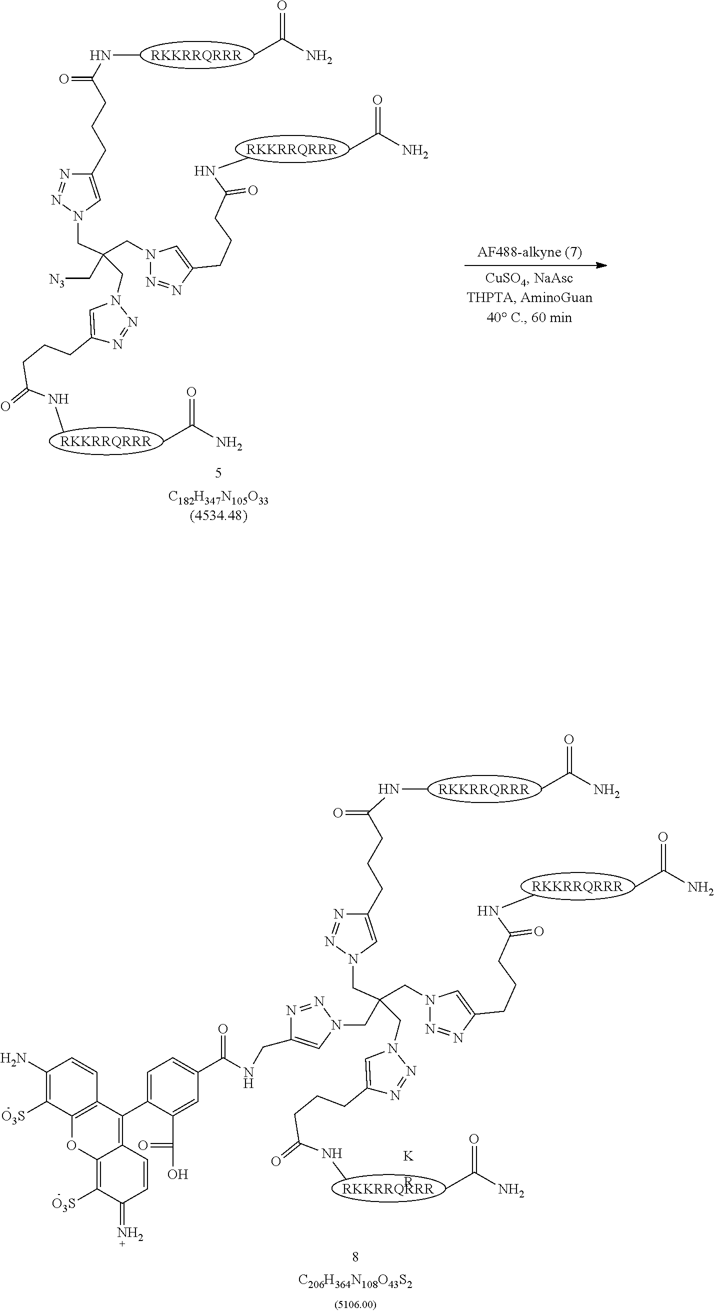

### tri-Tat A (8)

Tat₃-N₃ (5) (175 μg, 0.038 μmol, 1 eq.), AlexaFluor™488 5-carboxyamido(propargyl), bis(triethylammonium) salt, 5-isomer (7-AF488-alkyne) (Thermo Fisher Scientific, USA) (60 μg, 0.08 μmol, 2 eq.), as well as CuAAC regents were dissolved in 30% DMSO/PBS and heated at 40° C. for 60 min. The product was purified by HPLC, (33 μg, 0.007 μmol, 18% yield). HPLC retention time rₜ=18:04 min; MS QTOF [ES+]: 5106.37 (calculated: 5106.00 for C₂₀₆H₃₆₄N₁₀₈O₄₃S₂)

### Synthesis Compound 9

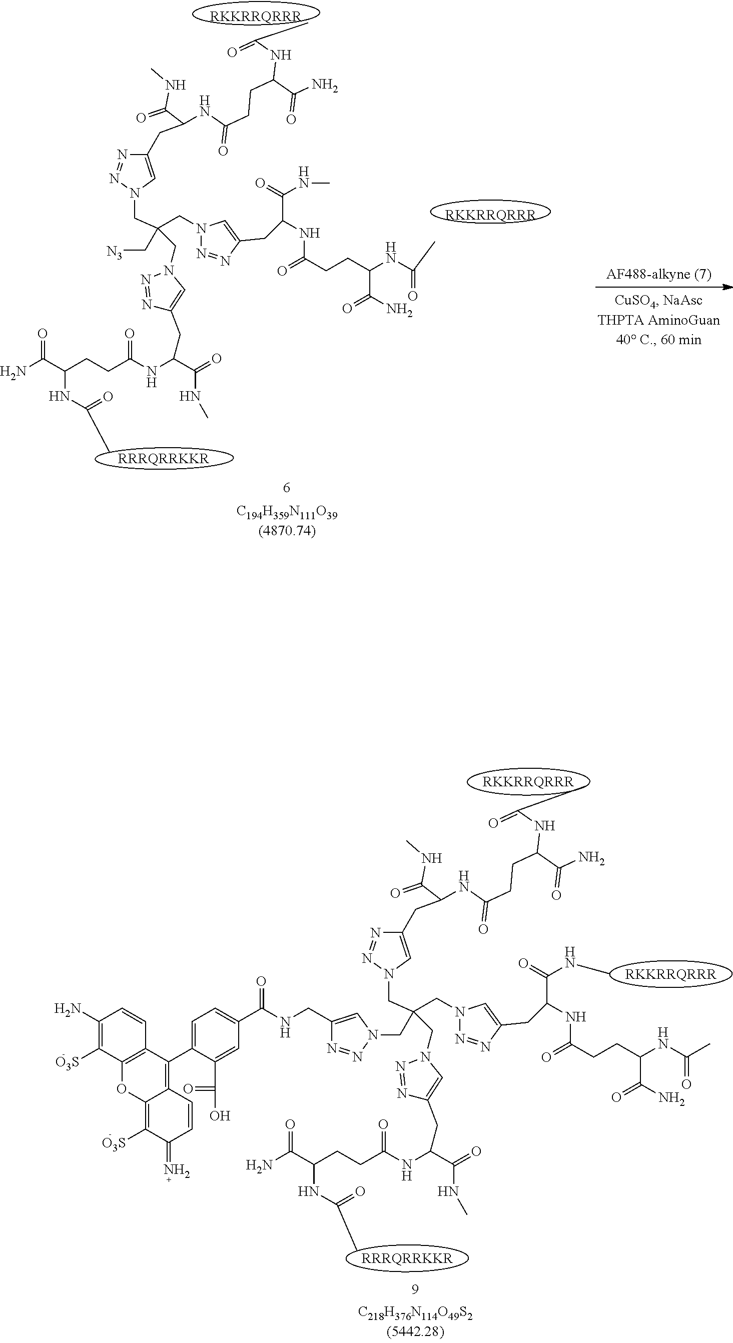

### tri-cTat A (9)

cTat₃-N₃ (6) (100 μg, 0.02 μmol, 1 eq.), AlexaFluor™488 5-carboxyamido(propargyl), bis(triethylammonium) salt, 5-isomer (7—AF488-alkyne) (Thermo Fisher Scientific, USA) (31 μg, 0.04 μmol, 2 eq.), as well as CuAAC regents were dissolved in 30% DMSO/PBS and heated at 40° C. for 60 min. The product was purified by HPLC, (35 μg, 0.006 μmol, 32% yield). HPLC retention time rₜ=18:47 min; MS QTOF [ES+]: 5443.27 (calculated: 5442.28 for C₂₁₈H₃₇₆N₁₁₄O₄₉S₂).

### Synthesis Compound 13

### Tat3-alkyne (13)

Tetrakis(2-propynyloxymethyl)methane (10) (13.4 μg, 0.05 μmol, 1 eq.), and 5-azido-pentanoyl-Tat-NH₂ (11—Tat-azide (49-57)) (Peptide International Inc., USA) (410 μg, 0.28 μmol, 6 eq.) as well as CuAAC regents were dissolved in 30% DMSO/PBS and heated at 40° C. for 60 min. The product was purified by HPLC (135 μg, 0.029 μmol, 61% yield).

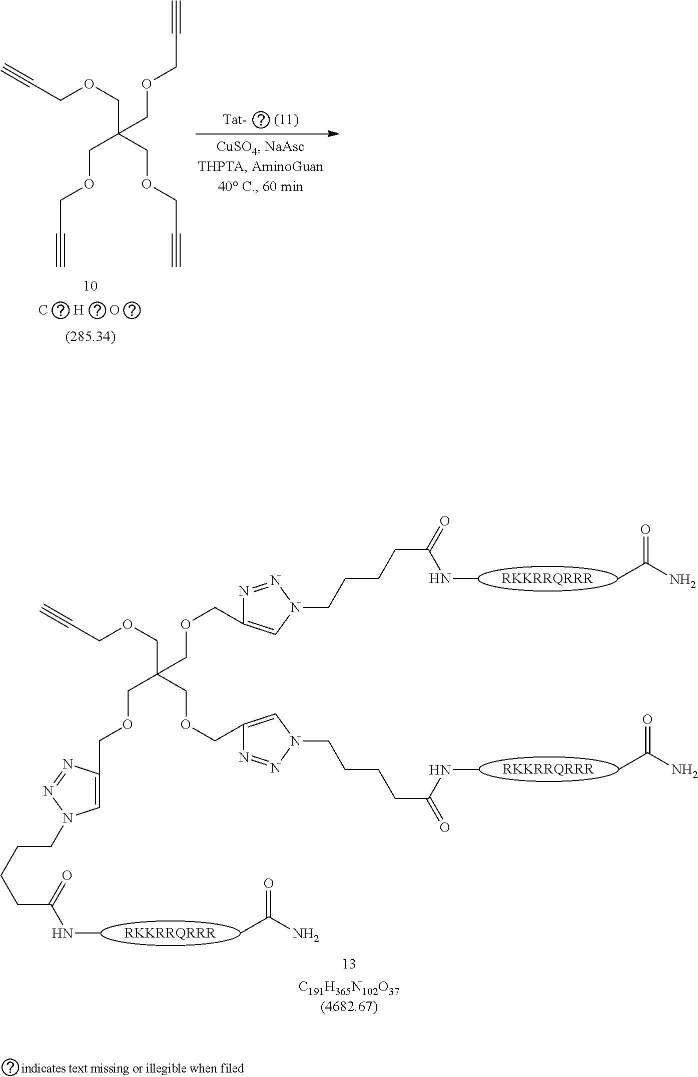

HPLC retention time rₜ=20:13 min; MS QTOF [ES+]: 4683.71 (calculated: 4682.67 for C₁₉₁H₃₆₅N₁₀₂O₃₇)

### Synthesis Compound 14

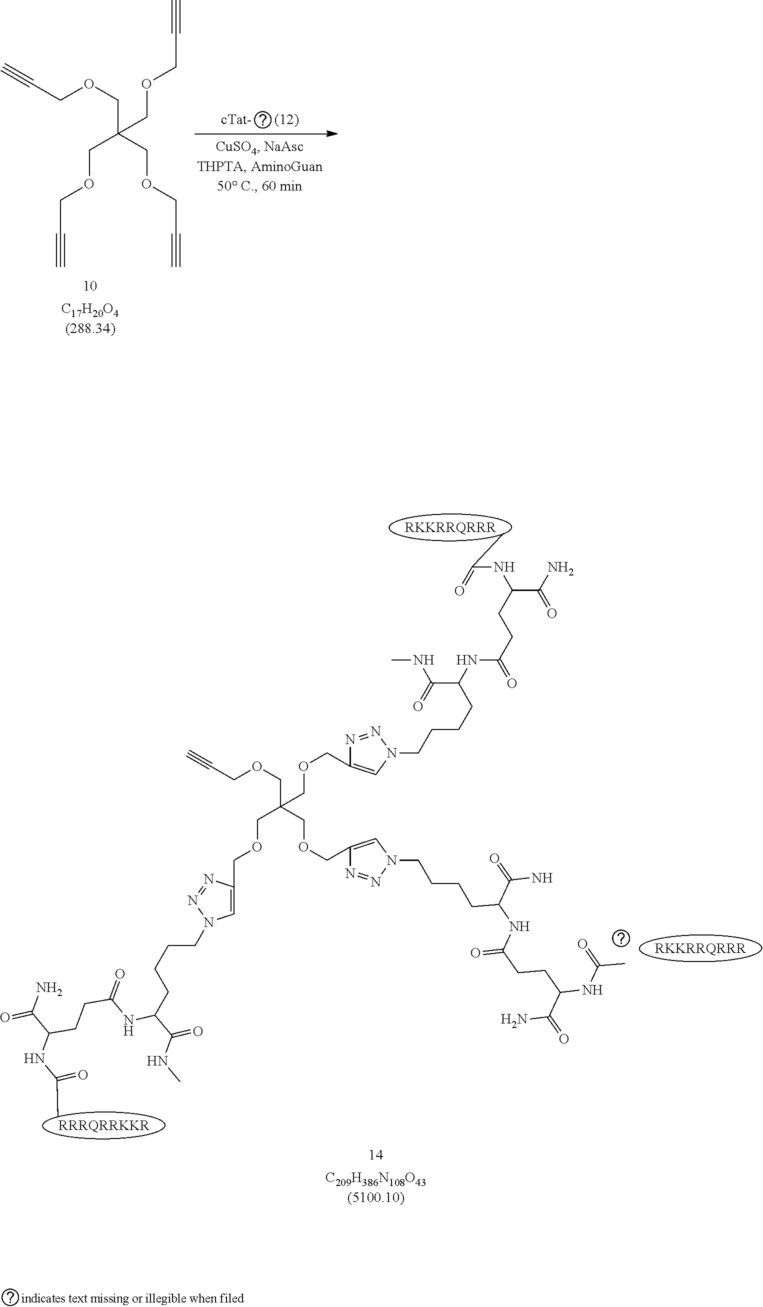

### cTat₃-alkyne (14)

Tetrakis(2-propynyloxymethyl)methane (10) (28.3 μg, 0.10 μmol, 1 eq.), and azidolysine-cyclicTat-NH₂ (12—cTat-azide (49-57)) (Cambridge Peptide, UK) (675 μg, 0.42 μmol, 4.2 eq.) as well as CuAAC regents were dissolved in 30% DMSO/PBS and heated at 50° C. for 80 min. The product was purified by HPLC (255 μg, 0.05 μmol, 51% yield).

HPLC retention time rₜ=20:12 min; MS QTOF [ES+]: 5100.32 (MW calculated: 5100.10 for C₂₀₉H₃₈₆N₁₀₈O₄₃)

### Synthesis Compound 16

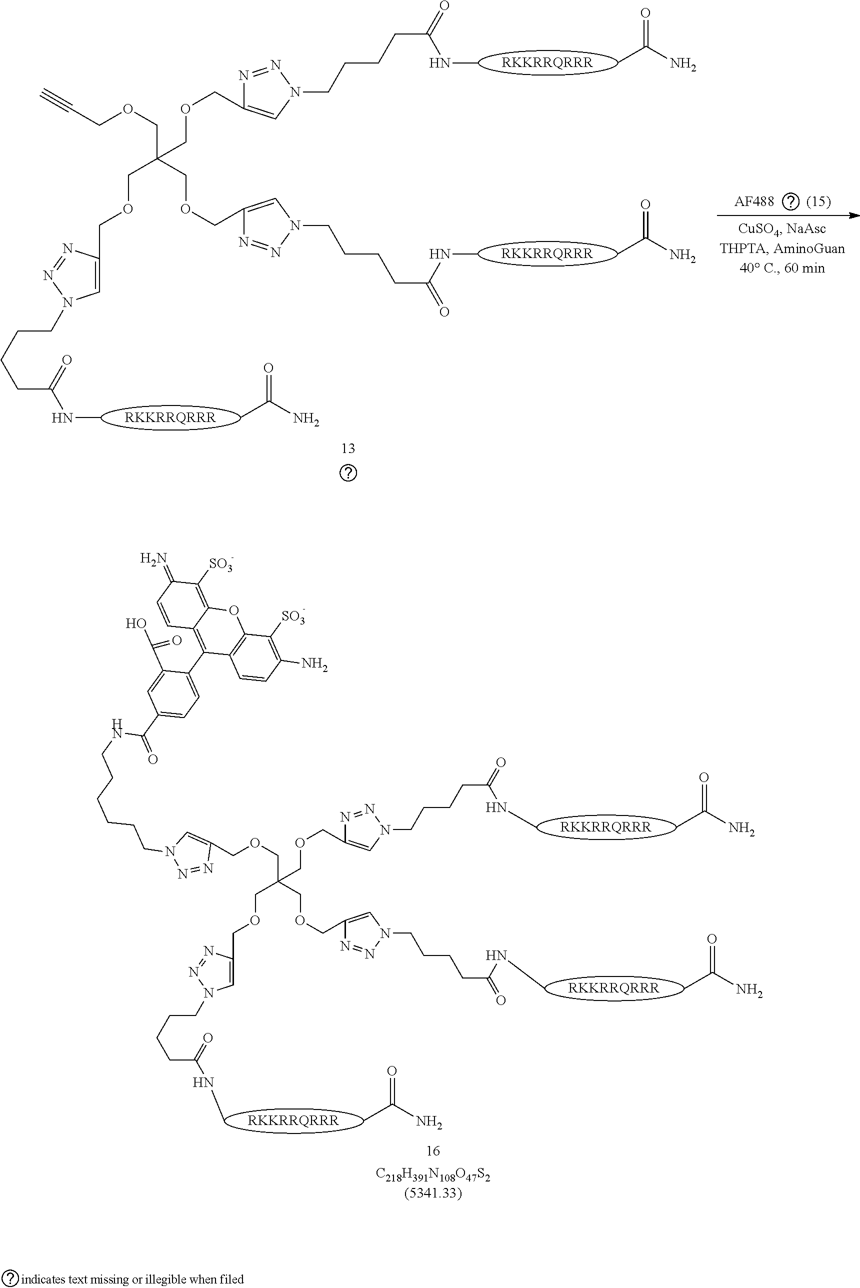

### tri-Tat B (16)

Tat₃-alkyne (13) (135 μg, 0.029 μmol, 1 eq.), Alexa Fluor™ 488 5-Carboxamido-(6-Azidohexanyl), Bis(Triethylammonium Salt), 5-isomer (15-AF488-azide) (Thermo Fisher Scientific, USA) (45 μg, 0.06 μmol, 2 eq.), as well as CuAAC regents were dissolved in 30% DMSO/PBS and heated at 40° C. for 60 min. The product was purified by HPLC (55 μg, 0.01 μmol, 36% yield).

HPLC retention time rₜ=22:43 min; MS QTOF [ES+]: 5342.18 (calculated: 5341.33 for C₂₁₈H₃₉₁N₁₀₈O₄₇S₂).

### Synthesis Compound 17

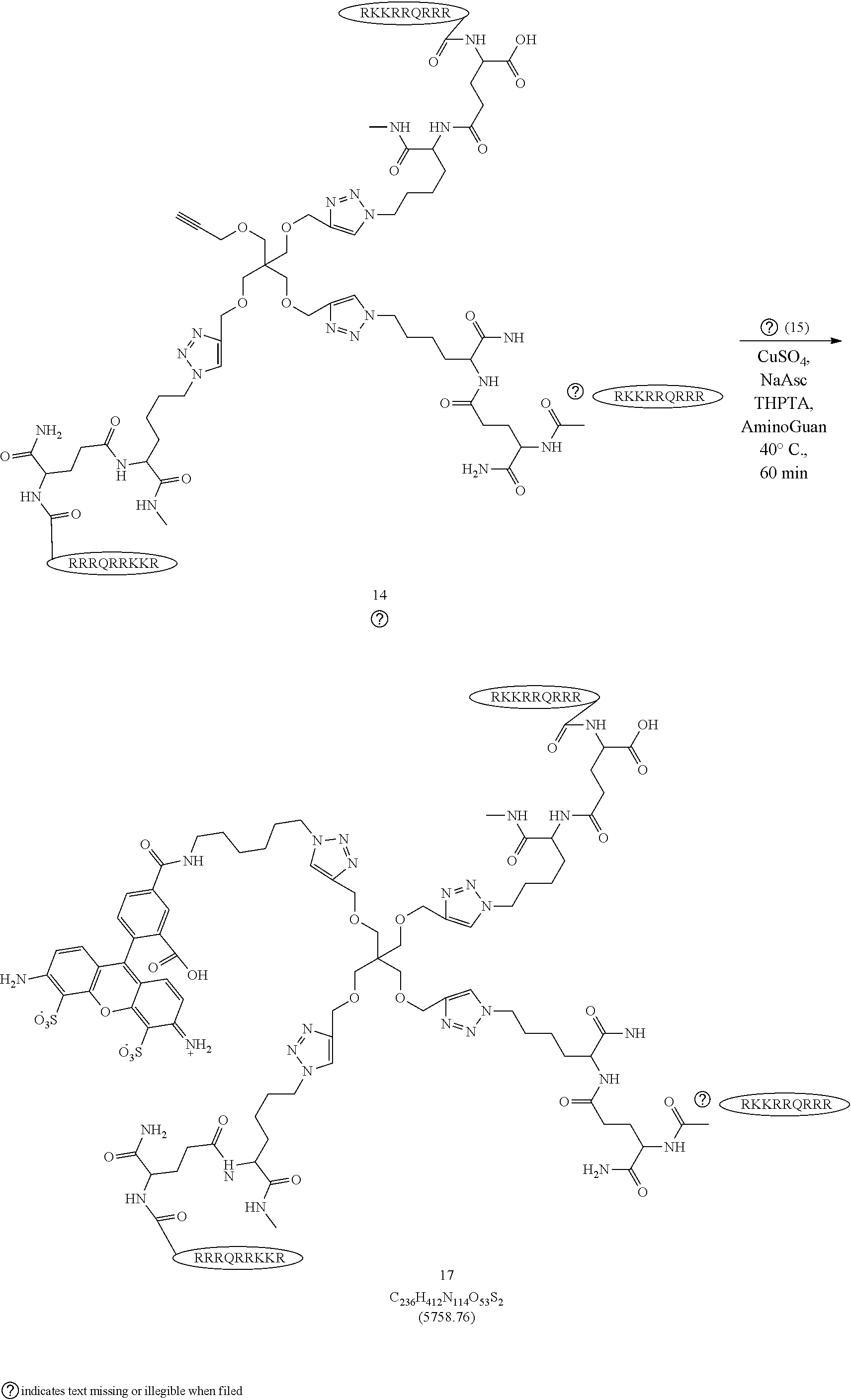

### tri-cTat B (17)

cTat₃-alkyne (14) (255 μg, 0.05 μmol, 1 eq.), Alexa Fluor™ 488 5-Carboxamido-(6-Azidohexanyl), Bis(Triethylammonium Salt), 5-isomer (15—AF488-azide) (Thermo Fisher Scientific, USA) (75 μg, 0.10 μmol, 2 eq.), as well as CuAAC regents were dissolved in 30% DMSO/PBS and heated at 40° C. for 60 min. The product was purified by HPLC (130 μg, 0.022μmol, 45% yield).

HPLC retention time rₜ=21:07 min; MS QTOF [ES+]: 5759.09 (MW calculated: 5758.76 for C₂₃₆H₄₁₂N₁₁₄O₅₃S₂).

### Synthesis Compound 18

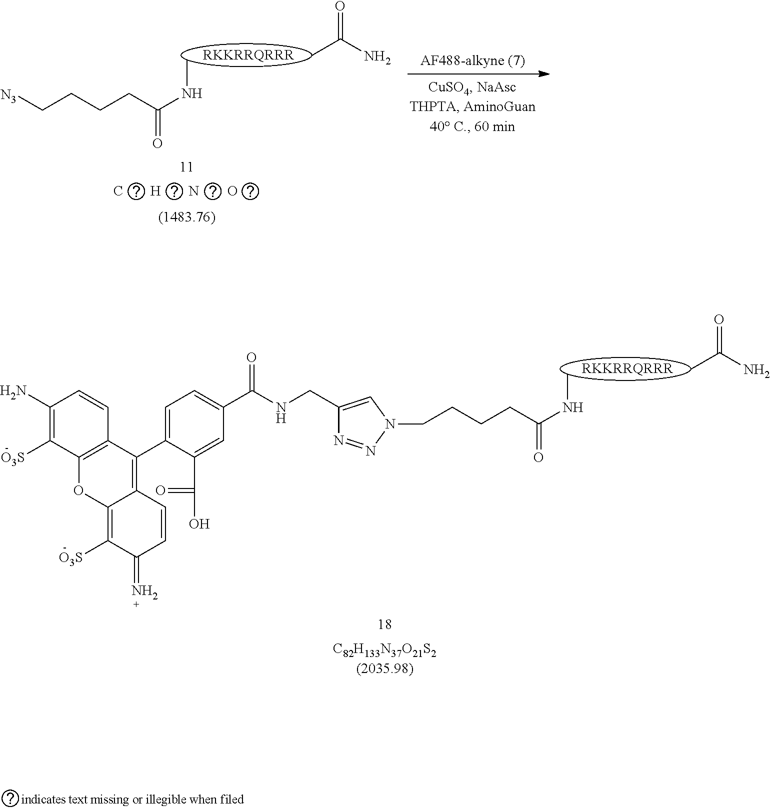

### mono-Tat (18)

5-azido-pentanoyl-Tat-NH₂ (3-Tat-azide (49-57)) (Peptide International Inc., USA) (205 μg, 0.14 μmol, 1 eq.), AlexaFluor™488 5-carboxyamido(propargyl), bis(triethylammonium) salt, 5-isomer (5—AF488-alkyne) (Thermo Fisher Scientific, USA) (162 μg, 0.21 μmol, 1.5 eq.), as well as CuAAC regents were dissolved in 30% DMSO/PBS and heated at 40° C. for 60 min. The product was purified by HPLC, (26 μg, 0.014 μmol, 10% yield).

HPLC retention time rₜ=18:01 min; MS QTOF: 2035.94 (calculated: 2035.98 for C₈₂H₁₃₃N₃₇O₂₁S₂).

### Synthesis Compound 19

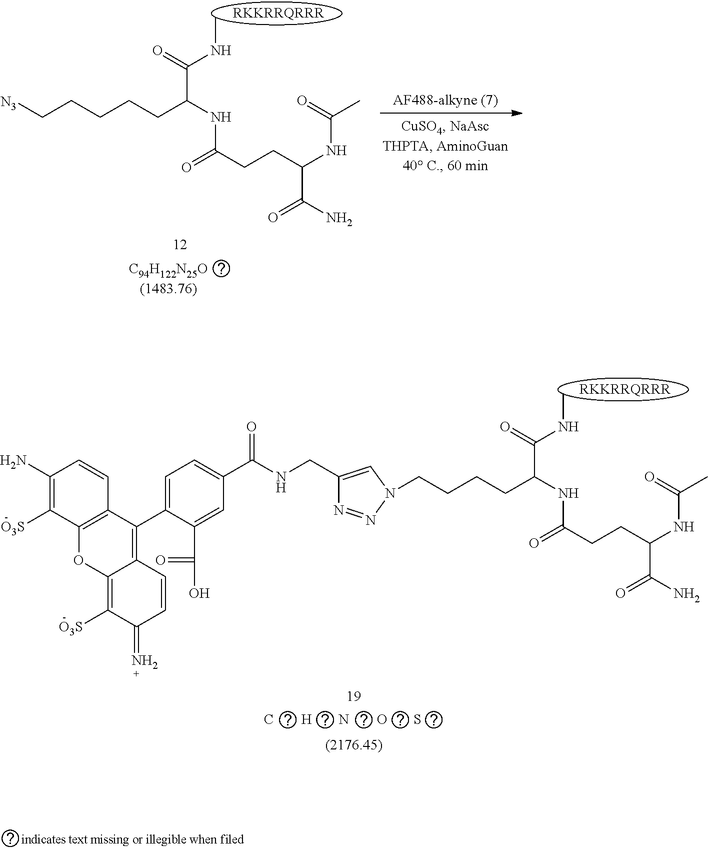

### mono-cTat (19)

Azidolysine-cyclicTat-NH₂ (12—cTat-azide (49-57) (Cambridge Peptide, UK) (225 μg, 0.14 μmol, 1 eq.), AlexaFluor™488 5-carboxyamido(propargyl), bis(triethylammonium) salt, 5-isomer (5—AF488-alkyne) (Thermo Fisher Scientific, USA) (162 μg, 0.21 μmol, 1.5 eq.), as well as CuAAC regents were dissolved in 30% DMSO/PBS and heated at 40° C. for 60 min. The product was purified by HPLC (75 μg, 0.034 μmol, 24% yield).

HPLC retention time rₜ=14:44 min; MS QTOF: 2075.03 (calculated: 2075.45 for C₈₈H₁₃₉N₃₉O₂₃S₂).

### General Procedure for Copper Catalyzed 2+3 Cycloaddition Reactions (Compounds 20-27)

Peptide and fluorophores were conjugated to the tetrameric PEG-alkyne scaffolds using copper-catalyzed azide alkyne cycloaddition reactions (CuAAC). Optimized reaction conditions include shaking at 1,000 rpm and heating at 40° C. for 60 to 240 min using a thermoshaker. Molar ratios of reagents were normalized to the number of CuAAC per molecule and are quoted for each reaction specifically. In general, the corresponding tetrameric PEG-alkyne scaffold (Tokyo Chemical Industry, 1 eq.) was dissolved in DMSO and thoroughly mixed with Tat-N₃ (11) or cTat-N₃ (12) peptides (Cambridge Peptides, 3.6 eq.). In other vial, copper sulfate (4 eq.) and THTPA (20 eq.) were mixed and both vials incubated at room temperature for 5 min. Sodium ascorbate (8 eq.) and aminoguanidine hydrochloride (12 eq.) were added to the vial containing copper sulfate and THTPA and mixed. The PEG-alkyne scaffold and Tat-N₃ (11)/cTat-N₃ (12) peptide was added to the vial containing the click reagents and thoroughly mixed. DMSO was added (final concentration 30% v/v) followed by PBS 0.1 M pH 7.3 (final concentration 15-30% v/v). The reaction vial was heated to 50° C. with shaking for 1.5-2 h. The reaction crude was concentrated on centrifugal 3 kDa filters, washed with PBS and purified by semi-preparative HPLC using water 0.1% TFA/acetonitrile 0.1% TFA.

### General Procedure for Labelled Tat Conjugate Synthesis (Compounds 20-26)

The concentrated trimer-alkyne product obtained above (1 eq.) was diluted with DMSO (30% v/v final concentration) and mixed with AlexaFluor™488 azide (ThermoFisher, 1-1.2 eq.) dissolved in DMSO. In a separate vial, copper sulfate (2 eq.) and THTPA (10 eq.) were mixed and both vials incubated at room temperature for 5 min. Sodium ascorbate (4 eq.) and aminoguanidine hydrochloride (6 eq.) were added to the vial containing copper sulfate and THTPA. The trimer-fluorophore mixture was added to the click reagents, PBS 0.1 M pH 7.3 added (5-15% v/v final concentration) and the vial thoroughly mixed. The reaction was heated to 50° C. with shaking for 1.5-2 h. The reaction crude was concentrated on centrifugal 3 kDa filters, washed with PBS until the filtrate was clear and purified by semi-preparative HPLC using the same gradient as above. The final product was concentrated on 3 kDa centrifugal filters and stored at −20° C.

### Synthesis Compound 20Tri-Tat C

Tat-N₃ (11-2000 μg, 1.37 μmol) was reacted with C—[CH₂—(O—CH₂—CH₂—O—)₁—CH₂—CH₂—O—C≡CH]₄ (“tetrameric PEG₃-alkyne”; 219 μg, 0.34 μmol) according to the general procedure set out above. HPLC retention time rₜ=5:50 min; MS QTOF [ESI⁺]: 5031.94 (calculated: 5032.10 for C₂₀₇H₃₉₄N₁₀₂O₄₅).

The resultant tetrameric PEG₃-Tat₃-alkyne (45 nmol) was reacted with AlexaFluor-488 azide (42.7 μg, 45 nmol) according to the general procedure set out above. HPLC retention time rₜ=5:58 min; MS QTOF [ESI⁺]: 5690.59 (calculated: 5690.75 for C₂₃₄H₄₂₀N₁₀₈O₅₅S₂).

### Synthesis Compound 21

### Tri-Tat D

Tat-N₃ (11—2000 μg, 1.37 μmol) was reacted with C—[CH₂—(O—CH₂—CH₂—O—)₂—CH₂—CH₂—O—C≡CH]₄ (“tetrameric PEG₅-alkyne”; 339 μg, 0.34 μmol) according to the general procedure set out above. HPLC retention time rₜ=6:10 min; MS QTOF [ESI⁺]: 5384.54 (calculated: 5384.52 for C₂₂₃H₄₂₆N₁₀₂O₅₃).

The resultant tetrameric PEG₅-Tat₃-alkyne (45 nmol) was reacted with AlexaFluor-488 azide (46.5 μg, 54 nmol) according to the general procedure set out above. HPLC retention time rₜ=6:22 min; MS QTOF [ESI⁺]: 6043.32 (calculated: 6043.18 for C₂₅₀H₄₅₂N₁₀₈O₆₃S₂).

### Synthesis Compound 22

### Tri-Tat E

Tat-N₃ (11—2000 μg, 1.37 μmol) was reacted with C—[CH₂—(O—CH₂—CH₂—O—)₃—CH₂—CH₂—O—C≡CH]₄ (“tetrameric PEG₇-alkyne”; 460 μg, 0.34 μmol). HPLC retention time rₜ=6:35 min; MS QTOF [ESI⁺]: 5736.55 (calculated: 5736.95 for C₂₃₉H₄₅₈N₁₀₂O₆₁).

The resultant tetrameric PEG₇-Tat₃-alkyne (71 nmol) was reacted with AlexaFluor-488 azide (73.1 μg, 85 nmol) according to the general procedure set out above. HPLC retention time rₜ=6:44 min; MS QTOF [ESI⁺]: 6395.58 (calculated: 6395.60 for C₂₆₆H₄₈₄N₁₀₈O₇₁S₂).

### Synthesis Compound 23

### Tri-Tat F

Tat-N₃ (11—2000 μg, 1.37 μmol) was reacted with C—[CH₂—(O—CH₂—CH₂—O—)₄—CH₂—CH₂—O—C≡CH]₄ (“tetrameric PEG₉-alkyne”; 580 μg, 0.34 μmol). HPLC retention time rₜ=6:53 min; MS QTOF [ESI⁺]: 6089.17 (calculated: 6089.37 for C₂₅₅H₄₉₀N₁₀₂O₆₉).

The resultant tetrameric PEG₉-Tat₃-alkyne (71 nmol) was reacted with AlexaFluor-488 azide (93.0 μg, 107 nmol) according to the general procedure set out above. HPLC retention time rₜ=7:04 min; MS QTOF [ESI⁺]: 6748.17 (calculated: 6748.03 for C₂₈₂H₅₁₆N₁₀₈O₇₉S₂).

### Synthesis Compound 24

### Tri-cTat C

cTat-N₃ (12—1350 μg, 0.84 μmol) was reacted with C—[CH₂—(O—CH₂—CH₂—O—)₁—CH₂—CH₂—O—C≡CH]₄ (“tetrameric PEG₃-alkyne”; 49 μg, 0.23 μmol) according to the general procedure set out above. HPLC retention time rₜ=5:50 min; MS QTOF [ESI⁺]: 5031.94 (calculated: 5452.25 (calculated: 5452.52 for C₂₂₅H₄₁₈N₁₀₈O₅₁).

The resultant tetrameric PEG₃-cTat₃-alkyne (74 nmol) was reacted with AlexaFluor-488 azide (46.5 μg, 74 nmol) according to the general procedure set out above. HPLC retention time rₜ=5:50 min; MS QTOF [ESI⁺]: 6111.39 (calculated: 6111.18 for C₂₅₂H₄₄₄N₁₁₄O₆₁).

### Synthesis Compound 25

### Tri-cTat D

cTat-N₃ (12—1350 μg, 0.84 μmol) was reacted with C—[CH₂—(O—CH₂—CH₂—O—)₂—CH₂—CH₂—O—C≡CH]₄ (“tetrameric PEG₅-alkyne”; 231.3 μg, 0.23 μmol) according to the general procedure set out above. HPLC retention time rₜ=6:10 min; MS QTOF [ESI⁺]: 5805.01 (calculated: 5804.95 for C₂₄₁H₄₅₀N₁₀₈O₅₉).

The resultant tetrameric PEG₅-cTat₃-alkyne (51 nmol) was reacted with AlexaFluor-488 azide (44.0 μg, 51 nmol) according to the general procedure set out above. HPLC retention time rₜ=6:18 min; MS QTOF [ESI⁺]: 6463.83 (calculated: 6463.60 for C₂₆₈H₄₇₆N₁₁₄O₆₉S₂).

### Synthesis Compound 26

### Tri-cTat E

cTat-N₃ (12—1350 μg, 0.84 μmol) was reacted with C—[CH₂—(O—CH₂—CH₂—O—)₃—CH₂—CH₂—O—C≡CH]₄ (“tetrameric PEG₇-alkyne”; 313.4 μg, 0.23 μmol). HPLC retention time rₜ=6:36 min; MS QTOF [ESI⁺]: 6157.49 (calculated: 6157.37 for C₂₅₇H₄₈₂N₁₀₈O₆₇).

The resultant tetrameric PEG₇-cTat₃-alkyne (24 nmol) was reacted with AlexaFluor-488 azide (21.0 μg, 24 nmol) according to the general procedure set out above. HPLC retention time rₜ=6:43 min; MS QTOF [ESI⁺]: 6816.46 (calculated: 6816.03 for C₂₈₄H₅₀₈N₁₁₄O₇₇S₂).

### Live Cell Confocal Microscopy

HeLa and CHO cells were cultured in DMEM supplemented with 10% FBS and penicillin/streptomycin. For post-wash experiments, Tat conjugate was added to the cells in serum free medium, cells incubated at 37° C. in an atmosphere of 5% CO₂, culture medium removed at the desired time point, cells washed and transferred to the microscope for imaging. For time course or pre-wash experiments, cells were transferred to the microscope (incubation chamber at 37° C., 5% CO₂) and Tat conjugate added directly to cell medium and cells imaged at desired time points. Co-delivery experiments were conducted by adding peptide trimer immediately followed by cargo into serum free medium. Samples were visualised using a Zeiss LSM 780 inverted confocal microscope fitted with a XLmultiS1 incubation chamber and Plan-Apochromat 63×/1.40 Oil DIC M27 objective. Images were analysed using ImageJ software. Digital adjustments and image processing are consistent throughout the manuscript, unless otherwise stated in the figure legend. Statistical analysis was carried out using GraphPad Prism 7 software and all data used for statistical analysis consists of measurements from distinct cells. Where applicable a parametric, unpaired, two-tailed t-test was used for statistical testing.

1. A compound comprising formula I: 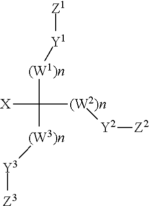
wherein;
W is an extension moiety,
X is selected from a cargo moiety, or H,
Y is a linking moiety,
Z is a cell penetrating peptide comprising SEQ ID NO:1 (RXXRRXRRR),
wherein n=0 to 10, and wherein Z is joined to Y via one atom of Z, or is joined to Y via at least two atoms of Z such as to form a cyclic moiety with Y.

2. The compound of claim 1, wherein the cell penetrating peptide comprises SEQ ID NO:2 (RKKRRQRRR).

3. The compound of claim 1, wherein the cell penetrating peptide is linear, or wherein the cell penetrating peptide is cyclic.

4. (canceled)

5. The compound of claim 3, wherein the cell penetrating peptide is cyclised via a C terminal glutamic acid and a N-terminal azide-modified lysine (K(N)₃) or propagyl glycine (Pra).

6. The compound of claim 1, wherein n=0 to 5.

7. The compound of claim 1, wherein the extension moiety is a hydrophilic polymer, optionally wherein the extension moiety is selected from (—CH₂—), (—CH₂—O—CH₂—), poly(glycerols) (PGs), poly(oxazolines) (POX), poly(hydroxypropyl methacrylate) (PHPMA), poly(2-hydroxyethyl methacrylate) (PHEMA), poly(N-(2-hydroxypropyl) methacrylamide) (HPMA), poly(vinylpyrrolidone) (PVP), poly(N,N-dimethyl acrylamide) (PDMA), poly(N-acryloylmorpholine) (PAcM), hyaluronic acid (HA), Heparin, or polysialic acid (PSA), preferably wherein the extension moiety is selected from (—CH₂—), or (—CH₂—O—CH₂—).

8. (canceled)

9. The compound of claim 7, wherein the extension moiety is (—CH₂—) and n=1 to 5, or wherein the extension moiety is (—CH₂—O—CH₂—) and n=1 to 5.

10. (canceled)

11. The compound of claim 1, wherein the linking moiety comprises a C3-C12 cycloalkyl, C3-C12 heterocycloalkyl, a C3-C12 aryl, a 3-membered to 12-membered heteroaryl, optionally wherein the linking moiety comprises an optionally substituted cycloalkyl, an optionally substituted heterocycloalkyl an optionally substituted aryl or an optionally substituted heteroaryl, wherein each cycloalkyl, heterocycloalkyl, aryl or heteroaryl is optionally substituted with one or more groups, selected from C1-C6 alkyl, C1-C6 carbonylalkyl, C1-C6 hydroxyalkyl, C1-C6 alkoxy or C1-C6 alkyl aminocarbonyl.

12. (canceled)

13. The compound of claim 1, wherein the linking moiety comprises a heterocyclic group, selected from a four-membered ring, a five membered ring, or a six membered ring, optionally wherein the linking moiety comprises 1 to 5 hetero atoms.

14. (canceled)

15. The compound of claim 1, wherein X comprises a cargo moiety wherein the cargo moiety is selected from a fluorophore, a small molecule therapeutic, a therapeutic peptide, a therapeutic protein, an antibody, an antigen-binding fragment, a single chain variable fragment, a single domain antibody, antibody fragment, or an oligonucleotide, optionally wherein the cargo moiety further comprises an extension moiety and/or a linking moiety.

16-20. (canceled)

21. A method of delivering a molecule into the cytosol of a cell comprising contacting a cell with the compound of claim 1.

22. The method of claim 21, wherein the molecule is covalently linked to the compound, or wherein the molecule is not covalently linked to the compound.

23. The method of claim 21, wherein the method is performed on a cell in vitro, ex vivo or in vivo.

24. A method of producing a cyclic Tat peptide comprising the steps of;
incorporating an azide modified lysine residue or a propagyl glycine to the N-terminus of the Tat peptide,
incorporating a glutamic acid to the C-terminus of the Tat peptide,
and performing head-to-tail cyclisation.

25. The method according to claim 24, wherein the Tat peptide comprises SEQ ID NO:1 (RXXRRXRRR), optionally wherein the Tat peptide comprises SEQ ID NO:2 (RKKRRQRRR).

26. An immunoconjugate comprising a compound according to claim 1 and a therapeutic moiety, optionally wherein the therapeutic moiety is an antibody or fragment thereof.

27. A pharmaceutical composition comprising a compound according to claim 1.

28. A kit comprising a compound according to claim 1.

29. A method of treatment comprising administration to a subject in need thereof, a compound according to claim 1.

30. The method of claim 21. wherein the molecule that is delivered to said cell is selected from an antibody, an antibody fragment or an oligonucleotide.
